(19) United States
(12) Patent Application Publication
(10) Pub. No.: US 2022/0373439 A1
(43) Pub. Date: Nov. 24, 2022

(54) BIOLOGICAL SAMPLE PURIFICATION APPARATUS, USE OF THE SAME, AND SYSTEMS COMPRISING THE SAME

(71) Applicants: ANATRACE PRODUCTS, LLC, Maumee, OH (US); PROTEIN ARK, LTD., Sheffield (GB)

(72) Inventors: MICHAEL DANIEL BROWNLEADER, Sheffield (GB); SIMON WILLIAM MAURICE TANLEY, Sheffield (GB)

(21) Appl. No.: 17/761,966

(22) PCT Filed: Sep. 18, 2020
(86) PCT No.: PCT/US2020/051474

§ 371 Date: Mar. 18, 2022

(30) Foreign Application Priority Data
Sep. 20, 2019 (GB) GB1913558.1

(51) Int. Cl. G01N 1/34; G01N 1/40; C12M 1/00

(52) U.S. Cl. CPC G01N 1/34; G01N 1/4005; G01N 1/405; G01N 1/4055; C12M 47/12; G01N 2001/4016

(57) ABSTRACT

A biological sample purification apparatus is described for purifying a protein from a cell, as well as methods of use of the purification apparatus, and systems comprising the same. The described apparatus comprises a housing comprising a top opening, a bottom opening, and a membrane positioned between said top opening and said bottom opening; and a purification media comprising diatomaceous earth and a resin, wherein the purification media is positioned between the membrane and the top opening; and wherein the purification media is optionally mixed and is substantially dry.

### BACKGROUND

Modern biotechnology revolves around not only the study of cells and cellular biology, but also the study of proteins. Proteins are comprised of covalent amino acid chains that function not only as catalysts for reactions, but also as key intercellular and intracellular biochemical signaling molecules, structural components of cell walls, cytoskeleton components, nucleocapsids, and the like. Proteins are found both embedded in, covalently attached to, and associated with cell membranes, as well as unattached monomers or multimers present in the cytoplasm or nucleus of cells, or in the extracellular matrix of biological organisms. The study of such proteins has led to a myriad of diagnostic tests, therapeutic treatments, and contributed to our basic understanding of life. However, to study such proteins, it is often required that they be analyzed in isolation, outside of the influence of other proteins and factors. Thus, purification of proteins is often the core of the beginning of any new biotechnology, diagnostic, or clinical therapy.

Methods of purification of proteins from biological samples have been evolving for decades and in classical biochemistry often involve chromatography, dialysis, precipitation, and various modes of protein detection. Newer biotechnology techniques have enabled determination of protein sequences, often by methods involving expression of recombinant proteins from autologous expression vectors transfected or transformed into host cells that are triggered to mass produce the recombinant protein based on an inducible signal. Even newer technologies have evolved that employ the concept of mass production and has led to automated, robotically controlled, high throughput expression, purification, and screening of proteins.

Yet, there still exists in these fields a dire need to continue to provide fast, efficient, clean, and simple methods for purifying proteins for study. Many of the latest protein purification technologies still require time consuming steps, requiring numerous resources, human intervention and activity, and often create enormous hazardous waste burdens.

To this end, described herein is a simple, efficient, cost-effective, and clean biological sample purification apparatus that is deployable as part of an automated high throughput system for studying single proteins or simultaneously isolating and screening large numbers of proteins. The described purification apparatus represents a sea change in chromatography purification processes because the apparatus can be mass produced and shipped at low cost containing no liquids, i.e. as a dry apparatus. Further, performance of the described purification apparatus is surprisingly effective for its simplicity. The apparatus purifies proteins from a crude cell preparation, as well as a crude cell lysate, or clarified cell lysate, or from complex mixtures of proteins, in a single, simple step. All that is required is addition of the sample to the housing comprising the described purification media, along with a source of liquid, and elution. The purification media comprises diatomaceous earth (DE), resin, and a membrane to prevent the DE and resin from draining out of the housing. The described apparatus is drip-free, clog-free, has a high capacity for sorting complex mixtures of biological materials, combines filtration and purification in a single step, and is amenable to automation and high-throughput protocols and applications.

### SUMMARY

Described herein is a biological sample purification apparatus useful for purifying one or more components, such as, but not limited to, proteins and/or cellular organelles, or cellular fractions from one or more biological samples. The sample, for instance, is from eukaryotic or prokaryotic source and either expresses a protein component to be purified either recombinantly or natively. The protein to be purified is in one embodiment secreted from a mammalian cell due to the presence of a secretion single encoded in the expression vector encoding the recombinant protein to be purified. In another embodiment, the protein is expressed recombinantly in a bacterial cell and the cell is lysed, and optionally partially or fully clarified prior to purification by way of the described purification devices. In another embodiment, the sample is a mixture of proteins and the operator of the purification apparatus desired to separate one type of protein from the mixture of proteins. Optionally, the proteins are soluble proteins.

Thus, in a first aspect provided is a purification apparatus for purifying a component from a biological sample, said apparatus comprising a housing comprising a top opening, a bottom opening, and a membrane positioned between said top opening and said bottom opening, and a purification media comprising diatomaceous earth (DE) and a resin, wherein the purification media is positioned between the membrane and the top opening, and wherein the purification media is substantially dry.

The purification apparatus described herein is suitable for purifying a component from a biological sample, i.e. the apparatus is a biological sample purification apparatus. The described apparatus comprises a housing comprising a top opening and a bottom opening. The top opening of said housing is configured to receive a biological sample. The bottom opening of said housing is configured to permit passage of components of the sample and/or the purification media out of the housing. Thus, the “top” opening is the housing inlet and the “bottom” opening is the housing outlet. In use, the purification media and sample components may pass through the housing, and out of the bottom opening due to the force of gravity and/or centrifugation and/or vacuum pressure and/or positive pressure. Thus, in use, the top opening is above the bottom opening. The membrane is positioned between the top and bottom openings, i.e. it is above the bottom opening of the housing (and below the top opening of the housing). The membrane is typically closer to the bottom opening (outlet) than it is to the top opening (inlet). The purification media is positioned above the membrane, i.e. between the top opening (inlet) and the membrane.

The membrane is configured to support the purification media when the housing is oriented in a vertical direction with the top opening above the bottom opening, i.e. the purification media contacts the upper face of the membrane and is retained in the housing by the membrane. Alternatively viewed, the membrane is configured to prevent passage of the purification media, and any other substances within the housing, through the bottom opening (outlet) of the housing in the absence of centrifugation or the application of vacuum force to the bottom opening or positive air pressure to the top opening. At the same time, the membrane is configured to permit passage of the purification media through the bottom opening of the housing upon centrifugation and/or application of vacuum force to the bottom opening or application of positive air force to the top opening.

The housing is in one embodiment within the context of a microtiter plate, i.e. a 24-well, 96-well, 384-well, or 1536-well plate, or an 8-well strip. In a particular embodiment, the housing is a well in a 96-well microtiter plate. In another embodiment, the housing is an 24-well microtiter plate. In another particular embodiment, the microtiter plate is otherwise known as a filter plate. In another embodiment, the housing is in the context of a spin column or other similar disposable/consumable column or tube made from a plastic polymer. The overall dimensions or size of the housing is not particularly limited, but in one embodiment the housing holds a total fluid volume of from about 0.6 mL to about 2.0 mL. In one embodiment, the housing tapers to a tip at the bottom opening below the membrane.

The purification apparatus comprises a purification media. The purification media comprises diatomaceous earth (DE) and a resin. In one embodiment, the DE and resin are comprised within a single layer, i.e., within the housing and above the membrane there is a layer of purification media comprising both DE and resin, i.e. a mixture of DE and resin. This layer is preferably homogenous, i.e. uniform in composition, i.e. in this layer the DE and resin are preferably thoroughly mixed. This layer is preferably in direct contact with the membrane. In some embodiments the purification media optionally includes a wetting agent.

In an alternative embodiment, the purification media comprises two layers, one layer comprising DE and the other layer comprising resin, one layer being positioned above the other within the housing, and both layers being positioned above the membrane. In other words, in this embodiment the purification media comprises an upper layer and a lower layer, the lower layer preferably being in direct contact with the membrane. The upper layer is closer to the top opening (inlet) and the bottom layer is closer to the bottom opening (outlet). The lower layer is preferably in direct contact with the membrane. Given the direction of passage of the sample within the housing when the apparatus is in use, the sample being inserted into the housing via the top opening (inlet), the upper layer of the purification media can be considered the first layer, and the lower layer can be considered the second layer. Particularly preferably, the upper layer comprises DE and the lower layer comprises resin. Alternatively, however, the upper layer may comprise resin and the lower layer may comprise DE. Further alternatively, the upper layer and the lower layer may both comprise both DE and resin, but one layer comprises a higher ratio of DE to resin, and the other comprises a higher ratio of resin to DE. In this embodiment, preferably the upper layer comprises a higher ratio of DE to resin, and preferably the lower layer comprises a higher ratio of resin to DE. Alternatively viewed, preferably the upper layer comprises more DE than the lower layer, and preferably the lower layer comprises more resin than the upper layer.

As explained elsewhere herein, in the preparation of the apparatus, the purification media components are optionally inserted into the housing via the top opening as wet components, e.g. in the form of a wet slurry. When the purification media comprises two layers as described above, the preparation of the apparatus optionally comprises placing a second wet component, e.g. a second wet slurry, on top of a first wet component, e.g. a first wet slurry within the housing, thereby forming the two media layers. In this scenario, it is possible that a small degree of mixing of the two layers occurs in the region where the two layers are in contact within the housing. This can be avoided, if desired, by careful application of the wet media layers and/or by drying the first media layer before applying the second wet layer. However, in many scenarios it is not essential to avoid such minor mixing of the layers. Thus optionally, the purification media comprises an interface region between the upper layer and the lower layer, said interface region comprising a mixture of DE and resin.

As is clear from the above description, the purification media is located inside the housing, forming a chromatography-type column arrangement whereby when in use, any liquid biological sample applied to the top of the housing contacts and passes through the purification media. The purification media is positioned above the membrane, preferably in direct contact with the membrane, and is thus above the bottom opening of the housing.

The purification media is substantially dry. As explained below, in the preparation of the purification apparatus, the purification media is optionally inserted into the housing in the form of wet components, e.g. wet slurries, and steps are then taken to dry the media. By “substantially dry” is meant that the media is essentially free of moisture. It does not mean that there is a strict literal requirement for media to be completely devoid of moisture, rather there is potential for a de minimis level of moisture to be present in the media even after steps have been taken to dry the media. A means for drying media may not be 100% effective, and a detailed inspection may reveal that some moisture is present. However, in a substantially dry media, moisture is present in such a small quantity that for the purposes intended and in terms of function of the purification apparatus the moisture can be considered absent. The skilled person can readily distinguish between a substance that is substantially dry and one that is not. Preferably, the purification media is completely dry.

In one embodiment, the resin is not an affinity resin. In another embodiment, the resin is affinity resin. In the affinity resin embodiment, the affinity resin is in some embodiments selected from one or more of Protein A, Protein G, Protein L, heparin, and lectin resin, or any known affinity resin. In a further embodiment the affinity resin comprises an antibody or antigen binding fragment thereof coupled to a resin support. Any known antibody or antigen binding fragment thereof that is amenable to coupling to resin while maintaining antigen binding activity can be used as the affinity resin. In further embodiments, the affinity resin comprises an immunoglobulin binding domain that binds an immunoglobulin Fc sequence and/or an immunoglobulin light chain sequence. In another embodiment, the affinity resin is an immobilized metal affinity chromatography (IMAC) resin. In one embodiment, when the affinity resin is an IMAC resin, the IMAC resin comprises one or more heavy metals, such as, but not limited to: Zn²⁺, Cu²⁺, Cd²⁺, Hg²⁺, Co²⁺, Ni²⁺, and Fe²⁺.

Any type of DE may be included in the purification media. Preferably, the type of DE included in the purification apparatus is acid washed DE. The acid-washed DE, in one embodiment, possesses a permeability of between about 0.025 and 1.000 Darcy (Darcy units are a measure of permeability where 1 Darcy permits a flow of 1 cm³ per second of a fluid that has a viscosity of 1 mPa·s or 1 cP under a pressure gradient of 1 atm/cm acting across an area of 1 cm²). In one embodiment, the DE comprises about 84.1 mg/kg aluminum, about 52.5 mg/kg calcium, about 50.5 mg/kg magnesium, about 20.0 mg/kg iron, about 10.5 mg/kg zinc, about 0.8 mg/kg copper, about 0.6 mg/kg antimony, about 0.7 mg/kg manganese, and about 0.2 mg/kg chromium. In another embodiment, the DE comprises about 6.2 mg/kg magnesium, about 2.8 mg/kg iron, about 0.6 mg/kg copper, and about 0.2 mg/kg manganese. In a particular embodiment, the DE comprises the following components at the indicated percentages:

| | % |
|---|---|
| SiO₂ | 98.65 |
| Al₂O₃ | 0.60 |
| Fe₂O₃ | 0.27 |
| Na₂O | 0.14 |
| K₂O | 0.10 |
| MgO | 0.08 |
| CaO | 0.08 |
| TiO₂ | 0.03 |
| P₂O₅ | 0.03 |

The acid-washed DE of the purification media may be produced by any known means and is within the competencies of the person of ordinary skill in the art. For instance, in one embodiment, the acid-washed DE is produced by washing the DE with water, then heating the washed DE to about 1000° C. to calcinate the DE, followed by washing the calcinated DE in acid to create acid-washed DE, and then heating the acid-washed DE in water to 200° C. to create dried, acid-washed DE.

The biological sample applied to the purification apparatus may comprise cells, e.g. mammalian cells. If so, then the preferred amount of DE included in the purification media can depend on the number of cells in the biological sample and/or whether or not the cells are lysed and whether or not the lysed cells have been clarified first before applying to the purification apparatus. Methods of determining the number of cells in a sample are standard in the field and within the competencies of the person of ordinary skill in the art. In one embodiment, the biological sample comprises eukaryotic cells, preferably mammalian cells. In such embodiments, preferably DE in the is included in the purification media in an amount of from about 1 mg DE per 6.25×10⁴ mammalian cells to about 1 mg DE per 1.25×10⁶ mammalian cells, preferably from about 1 mg DE per 24×10⁴ mammalian cells to about 1 mg DE per 37.5×10⁴ mammalian cells. The same amounts of DE are preferred if the sample comprises any eukaryotic cell type. When the sample comprises prokaryotic cells, e.g. bacterial cells, preferably the DE is present in an amount of less than approximately 1 mg DE per 2.5×10⁶ lysed cells.

As explained above, the membrane is configured to support the purification media and retain the purification media inside the housing. The membrane does not markedly or detectably interact with the sample or the component to be purified therefrom. Such membranes are well-known components of purification apparatuses. The person of ordinary skill in the art will be aware of such membranes and will readily be able to use a membrane suitable for his/her specific purposes. The membrane may be comprised of any material suitable for such purposes. Preferably, the membrane comprises PTFE. Preferably the membrane is 100% PTFE. In one embodiment, the membrane prevents the purification media from dropping through the housing and out the bottom opening when the purification apparatus is oriented in a vertical direction with the top opening above the bottom opening.

Also provided herein are processes for producing the described purification apparatus. Thus, in a further aspect, provided herein is a method of preparing a purification apparatus for purifying a component from a biological sample, said method comprising: A) providing a housing comprising a top opening, a bottom opening, and a membrane positioned between said top opening and said bottom opening; B) adding to the housing a purification media comprising diatomaceous earth (DE) and a resin, wherein the purification media is positioned above the membrane and between the top opening and the membrane; and C) drying the purification media.

The housing may be a housing described anywhere else herein.

Housings comprising a top opening, a bottom opening, and a membrane positioned between said top opening and said bottom opening are commercially available. Additionally, such housings without said membrane are available and it is within the competencies of the skilled person to add said membrane thereto. Thus, step A) optionally comprises providing a housing comprising a top opening and a bottom opening opposed thereto and adding the membrane to the housing via the top opening or bottom opening thereof.

Step B) preferably comprises one of the following steps: Bi) adding to the housing via the top opening thereof a composition comprising DE and a resin, preferably wherein said composition is wet, preferably a wet slurry. Preferably, the composition comprises a mixture of DE and resin, preferably a homogenous mixture of DE and resin; OR Bii) adding to the housing via the top opening thereof a composition comprising resin, followed by adding to the housing via the top opening thereof a second composition comprising diatomaceous earth (DE), wherein said compositions are wet, preferably wet slurries; OR Biii) adding to the housing via the top opening thereof a first composition comprising DE, followed by adding to the housing via the top opening thereof a second composition comprising resin, wherein said compositions are wet, preferably wet slurries.

Due to the presence of the membrane between the top and bottom openings of the housing, the insertion of the above-mentioned wet compositions (e.g. slurries) into the housing necessarily means that the resulting purification media within the housing is positioned above the membrane.

Steps Bii) and Biii) will each result in the formation within the housing of a purification media comprising two layers, i.e. an upper layer and a lower layer. The first wet composition will form the lower layer of the purification media such that it directly contacts the membrane, and the second wet composition will form the upper layer of the purification media.

In steps Bii) and Biii), preferably the additions of the first and second wet compositions are such that of the resulting two layers of purification media (the upper layer and the lower layer), one layer comprises DE and one layer comprises resin. Preferably, the first wet composition (forming the lower layer) comprises resin and the second wet composition (forming the upper layer) comprises DE.

Optionally, the first wet composition and the second wet composition both comprise DE and resin, but one wet composition comprises a higher ratio of DE to resin, and the other wet composition comprises a lower ratio of resin to DE. In this scenario, preferably the first wet composition comprises a higher ratio of resin to DE, and preferably the second wet composition comprises a higher ratio of resin to DE. Alternatively viewed, preferably the first wet composition comprises more resin than the second wet composition, and preferably the second wet composition comprises more DE than the first wet composition.

In steps Bii) and Biii), optionally the additions are such that essentially no mixing of the upper and lower layers occurs following the addition. Careful application of the second wet composition will minimize turbulence at the interface of the wet compositions, thereby avoiding mixing. Preferably, to avoid such mixing, the method comprises an additional step of drying the first composition after addition thereof to the housing but before addition of the second composition to the housing. Suitable drying steps are those described elsewhere herein.

Alternatively, in steps Bii) and Biii) the additions of the first and second wet compositions (e.g. slurries) are such that mixing of the two wet compositions is allowed to occur in the region where the two compositions are in contact (i.e. at the interface of the compositions). The result is a minor mixing of the compositions, i.e. the resulting purification media comprises an interface region between the upper layer and the lower layer, said interface region comprising a mixture of DE and resin.

Optionally, following step Bii) or Biii), the two wet compositions are thoroughly mixed either before addition to the housing or in the housing, e.g. by shaking the housing on a shaker, tilter, vortex, or other mechanical mixing device, to form a single layer of purification media comprising both DE and resin, preferably a mixture of DE and resin, preferably a homogenous mixture of DE and resin.

In one embodiment of this method, the first and second wet compositions are slurries, preferably 50:50 slurries, i.e. each comprising either 50% DE in distilled water or 50% fully hydrated resin, and an aqueous solution. The aqueous solution in some embodiments comprises water, an alcohol, and optionally a preservative.

Addition of components, e.g. media components and membrane components, to the housing may be performed using any known technique. Preferably, addition to the housing is by means of, for instance, a pipette. In one embodiment, this process is automated and liquid/slurry is handled by robotic arms equipped with pipetting instrumentation capable of delivering specific volumes of liquid/slurry into the housing.

As described above, the method comprises a drying step C) as well as optional drying steps in between addition of the purification media components to the housing. It is within the competencies of the person of ordinary skill in the art to design and use a drying step suitable for his/her purposes. Preferably, a drying step may comprise exposure of the purification media, wet composition, slurry, liquid, or any other component to ambient air at ambient temperature for a period of time, e.g. several hours, or overnight, or for several days, or may comprise application of heated air, i.e. air that is warmer than ambient air. The application of warmer air may accelerate drying. Preferably, the air is at a temperature that does not degrade or transform the housing, purification media, membrane, or any other component of the apparatus. Exposure to air that is warmer than ambient air is achieved, for example, by incubating the purification apparatus in an incubator or other mechanical apparatus equipped with heating capacity, i.e. a heating element, allowing exposure of the purification apparatus to constant, or increasing, elevated temperatures over a period of time. Preferably, the drying step is performed until the purification media is substantially dry.

Alternatively, the DE and resin are added to the housing in dry form. The DE and resin in dry form may be mixed prior to or subsequent to their addition to the housing, or alternatively not mixed at all prior to addition to the housing. In a further embodiment, the housing is sealed after production to prevent the purification media from falling out of the housing during shipping or transport. Such seals are well known and include any commercially available plastic wrap, paraffin, wax, foil, or similar composition able to attach to the top opening in a fixed manner precluding exit of the purification media from the housing.

Also described herein are uses of, and methods of using, the described purification apparatus. Such uses and methods of use include, for instance, purification of a target protein away from other components of a liquid sample mixture. Thus, in a further aspect, provided are methods of purifying a component from a biological sample, said method comprising: A) applying a biological sample to a top opening of a purification apparatus for purifying a component from a biological sample, wherein the purification apparatus comprises: a housing comprising a top opening, a bottom opening, and a membrane positioned between said top opening and said bottom opening; and a purification media comprising diatomaceous earth (DE) and a resin; wherein the purification media is positioned between the membrane and the top opening; and wherein the purification media is substantially dry; and B) mixing the sample with the purification media in the housing.

Methods and uses are described that comprise applying a biological sample to a top opening of the above-described purification apparatus. Preferably the biological sample is a liquid biological sample. Alternatively, however, the sample may be a dried sample and either before or after applying the biological sample to the apparatus, water and/or a buffered compatible solution is applied to hydrate the sample and the purification media prior to mixing.

After applying the biological sample to the top opening of the housing, and after hydrating said sample if it is a dried sample at that step, then the biological sample is mixed with the purification media in the housing (step B). In step B), the mixing is preferably such that the sample and the purification media are thoroughly mixed, i.e such that the sample and purification media form a single layer, are homogeneous, and/or have reached equilibrium such that all components of the sample have had sufficient time to come into contact with the DE and the resin in the purification media. In step B), mixing may be accomplished by shaking the housing on a shaker, tilter, vortex device, or other mechanical disruption device. In this step, the component to be separated from the biological sample becomes bound to, associated with, adhered to, coordinated with, or otherwise contacted with the resin in the purification media, thereby becoming purified from the biological sample through interaction with the resin.

Optionally, after step B), the method comprises a step of clearing liquid from the apparatus via the bottom opening. This liquid is termed “flowthrough” and may be cleared by gravity flow, centrifugation, application of a positive air pressure to the top opening of the apparatus, or application of a vacuum to the bottom opening.

Preferably, after step B), and after said clearing step if present, the method comprises one or more steps of washing said purification media. This step is performed in order to remove components still present in the apparatus that are not bound to, or otherwise associated with, the purification media. In embodiments in which whole cells are applied to the purification apparatus, whole cells remain on top of the purification media and do not flow through the housing, but otherwise do not block or clog the flow of liquid through the housing. Preferably, such washing steps are performed by adding a compatible buffered liquid media to the housing via the top opening thereof, and allowing the wash liquid to flow through the housing by gravity flow, centrifugation, application of a positive air pressure to the top opening, or application of a vacuum to the bottom opening of the housing. The washing steps may be performed once, twice, or any number of iterations, until all, or substantially all, of the unbound/unassociated components and/or contaminants are removed from the apparatus.

Preferably, after step B), and after said clearing liquid and washing step(s), if present, the method comprises a step of eluting the purified component from the apparatus. Preferably, such an eluting step is performed by adding a compatible liquid elution buffer to the housing via the top opening thereof, and letting the elution buffer flow through the housing by gravity flow, centrifugation, application of a positive air pressure to the top opening, or application of a vacuum to the bottom opening of the housing. Alternatively, the component may be eluted without application of elution buffer, merely by centrifugation or application of a vacuum to the bottom opening.

In all aspects of the present invention, the component to be purified from the biological sample may be any component of said sample. Preferably, the component is a nucleic acid molecule, protein, cell organelle, or cellular fraction. Most preferably the component is a protein, i.e. a target protein. Components to be purified are also referred to as “analytes” herein.

In all aspects of the described purification apparatus and the methods of use and uses thereof, the biological sample may be any sample comprising biological material, i.e. biological molecules. The sample may be a complex biological sample. The biological sample may be an unpurified sample, a crude cell preparation, a crude cell lysate, a whole cell extract, a clarified cell lysate, a clarified whole cell extract, a tissue homogenate, soil sample, water sample, sample of bodily fluid, plant extract, or other complex mixture of proteins. Cell extracts and lysates can be clarified by known processes of centrifugation or filtration and the like. In particular, the biological samples purified according to the methods described herein may comprise cells, including whole cells, lysed cells, cell components, and/or organelles, cell membrane structures, or a combination of these components. The cells may express a recombinant protein, optionally a secreted recombinant protein. Thus, the biological sample may comprise an expressed recombinant protein.

The component to be purified is preferably a protein. A “target protein” as used herein is a preferred component of interest, i.e. a component to be purified away from other non-preferred components, or contaminants, of said biological sample. The protein may be an expressed recombinant protein, as described above. The sample may comprise one or more contaminating proteins (or contaminants). In some embodiments the target protein is secreted from one or more cells in the sample, and in other embodiments the target protein is recombinantly expressed and must be obtained by first lysing the cells and optionally clarifying the cell lysate of all or substantially all of the cellular membrane components and other non-soluble cellular fractions. Preferably, the cells are eukaryotic or prokaryotic cells. Preferably the cells are selected from, for example, yeast cells, bacterial cells, mammalian cells, insect cells, human cells, plant cells, and the like. Preferably the cells are mammalian cells. Preferably the sample is a sample obtained from a subject or patient, preferably a human subject or patient.

In some embodiments, the target protein comprises a histidine linker, lectin, carbohydrate, or other affinity ligand, such as an antibody or antigen binding fragment thereof, allowing purification through the presence within the purification media of an affinity resin with a cognate binding partner attached thereto or associated therewith in a stable manner. In another embodiment, the sample comprises cells that overexpress the protein to be purified, and the target protein is the predominant protein in the biological sample. In a particular embodiment, the target protein comprises at least one immunoglobulin domain.

Disclosures and preferred and optional features and embodiments disclosed anywhere herein in relation to the described purification apparatus apply mutatis mutandis to the disclosures and preferred and optional features and embodiments disclosed herein in relation to the purification apparatus prepared or used in the methods of use thereof described herein. In other words, in the context of each and all methods of the present invention, the purification apparatus is preferably that described anywhere else herein.

This Summary is provided to introduce a selection of concepts in a simplified form that are further described below in the Detailed Description. This Summary is not intended to identify critical or essential features of the claimed subject matter, nor is it intended to fully limit the scope of the claimed subject matter described more fully hereinbelow.

### BRIEF DESCRIPTION OF THE DRAWINGS/FIGURES

For a more precise understanding of the disclosed apparatuses, systems comprising the same, and methods using the same, reference is made to specific embodiments thereof illustrated in the drawings. The drawings presented herein are not drawn to scale and any reference to dimensions in the drawings or the following description are with reference to specific embodiments. It will be clear to one of skill in the art that variations of these dimensions are possible while still maintaining full functionality for the intended purpose. Such variations are specifically contemplated and incorporated into this disclosure notwithstanding the specific embodiments set forth in the following drawings.

FIG. 1A and FIG. 1B provide units of green fluorescent protein (GFP) detected in wells of a 96-well microtiter plate as a control experiment. FIG. 1A depicts data from samples containing 600, 800, or 1000 μL of GFP solution and subjected to shaking at 600 rpm, whereas FIG. 1B depicts data from 600, 800, or 1000 μL of GFP solution subjected to shaking at 1000 rpm.

FIG. 2 provides a photograph of a protein stained and dried reducing SDS-PAGE gel showing proteins detected in the initial sample, the flowthrough, and the eluates of samples containing HEK cells into which recombinant His-tagged ULP protease was added. The first lane on the left shows Elite Pre-Stained Ladders. Samples were purified using Fastback Nickel Advance resin (Protein Ark, Ld., Sheffield, UK) with no DE present in the purification media. Sample 1 contained 2.5×10⁶ cells/mL. Sample 2 contained 5.0×10⁶ cells/mL. Sample 3 contained 10.0×10⁶ cells/mL. Sample 4 contained 25.0×10⁶ cells/mL. Sample 5 contained 50.0×10⁶ cells/mL.

FIG. 3 provides a photograph of a protein stained and dried reducing SDS-PAGE gel showing proteins detected in the initial sample, the flowthrough, and the eluates of samples containing HEK cells into which recombinant His-tagged ULP protease. The first lane on the left shows Elite Pre-Stained Ladders. Samples were purified using Fastback Nickel Advance resin (Protein Ark, Ld., Sheffield, UK) with DE present in the purification media. Sample 1 contained 2.5×10⁶ cells/mL. Sample 2 contained 5.0×10⁶ cells/mL. Sample 3 contained 10.0×10⁶ cells/mL. Sample 4 contained 25.0×10⁶ cells/mL. Sample 5 contained 50.0×10⁶ cells/mL.

FIG. 4 provides a photograph of a dried and protein-stained reducing SDS-PAGE gel with lanes numbered at the top corresponding to the following samples: i) 2.5×10⁶/mL, ii) 5.0×10⁶/mL, iii) 10.0×10⁶/mL, and iv) 25.0×10⁶/mL (“F” means flowthrough, and “E” means eluate). The dark bands in the middle of the gel correspond to purified His-tagged ULP protease expressed from insect Sf9 cells. In this figure, all samples were purified without the presence of DE.

FIG. 5 provides a photograph of a dried and protein-stained reducing SDS-PAGE gel with lanes numbered at the top corresponding to the following samples: i) 2.5×10⁶/mL, ii) 5.0×10⁶/mL, iii) 10.0×10⁶/mL, and iv) 25.0×10⁶/mL (“F” means flowthrough, and “E” means eluate). The dark bands in the middle of the gel correspond to purified His-tagged ULP protease expressed from insect Sf9 cells. In this figure, all samples were purified with the presence of DE.

FIG. 6 provides a bar graph of GFP fluorescence measurement data from Samples A, B, C, and D, obtained from E. coli cell lysates expressing His-tagged GFP. GFP fluorescence was measurement from the last wash as well as from the elution (dark bar graph is GFP measured in the last wash and the lighter bars indicate GFP detected in the elution).

FIG. 7 provides a photograph of a dried and protein-stained reducing SDS-PAGE gel with lanes numbered at the top corresponding to the following samples: 1) Elite Pre-Stained Ladders (Protein Ark, Ltd., Sheffield, UK); 2) 25×10⁶ cells/mL wash; 3) 25×10⁶ cells/mL elution; 4) 15×10⁶ cells/mL wash; 5) 15×10⁶ cells/mL wash; 6) 10×10⁶ cells/mL wash; 7) 10×10⁶ cells/ml elution; 8) 5×10⁶ cells/mL wash; 9) 5×10⁶ cells/mL elution; 10) 2.5×10⁶ cells/mL wash; and 11) 2.5×10⁶ cells/mL elution. Samples were taken from HEK mammalian cell cultures secreting recombinant IgG antibody. Purification media included Protein A affinity resin and no DE.

FIG. 8 provides a photograph of a dried and protein-stained reducing SDS-PAGE gel with lanes numbered at the top corresponding to the following samples: 1) Elite Pre-Stained Ladders (Protein Ark, Ltd., Sheffield, UK); 2) 25×10⁶ cells/mL wash; 3) 25×10⁶ cells/mL elution; 4) 15×10⁶ cells/mL wash; 5) 15×10⁶ cells/mL wash; 6) 10×10⁶ cells/mL wash; 7) 10×10⁶ cells/ml elution; 8) 5×10⁶ cells/mL wash; 9) 5×10⁶ cells/mL elution; 10) 2.5×10⁶ cells/mL wash; and 11) 2.5×10⁶ cells/mL elution. Samples were taken from HEK mammalian cell cultures secreting recombinant IgG antibody. Purification media included Protein A affinity resin with DE.

FIG. 9 provides a photograph of a dried and protein-stained reducing SDS-PAGE gel with lanes numbered at the top corresponding to the following samples:1) Elite Pre-Stained Ladders; 2) 25×10⁶ cells/mL wash; 3) 25×10⁶ cells/mL elution; 4) 15×10⁶ cells/mL wash; 5) 15×10⁶ cells/mL wash; 6) 10×10⁶ cells/mL wash; 7) 10×10⁶ cells/ml elution; 8) 5×10⁶ cells/mL wash; 9) 5×10⁶ cells/mL elution; 10) 2.5×10⁶ cells/mL wash; and 11) 2.5×10⁶ cells/mL elution. Samples were taken from CHO mammalian cell cultures secreting recombinant IgG antibody. Purification media included Protein A affinity resin and no DE.

FIG. 10 provides a photograph of a dried and protein-stained reducing SDS-PAGE gel with lanes numbered at the top corresponding to the following samples: 1) Elite Pre-Stained Ladders; 2) 25×10⁶ cells/mL wash; 3) 25×10⁶ cells/mL elution; 4) 15×10⁶ cells/mL wash; 5) 15×10⁶ cells/mL wash; 6) 10×10⁶ cells/mL wash; 7) 10×10⁶ cells/ml elution; 8) 5×10⁶ cells/mL wash; 9) 5×10⁶ cells/mL elution; 10) 2.5×10⁶ cells/mL wash; and 11) 2.5×10⁶ cells/mL elution. Samples were taken from CHO mammalian cell cultures secreting recombinant IgG antibody. Purification media included Protein A affinity resin with DE.

### DETAILED DESCRIPTION

### Definitions

The term “a” or “an” entity as used herein refers to one or more of that entity; for example, “a cell,” is understood to represent one or more cells. As such, the terms “a” (or “an”), “one or more,” and “at least one” are herein used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about” or “approximately” refers to a variation of 10% from the indicated values (e.g., 50%, 45%, 40%, etc.), or in case of a range of values, means a 10% variation from both the lower and upper limits of such ranges. For instance, “about 50%” refers to a range of between 45% and 55%.

As used herein, the term “analyte” refers to the component purified from (or to be purified from) the biological sample. The terms “analyte”, “component of interest” and “target component” are used interchangeably. Preferably the component of interest is a protein. The term “protein of interest” and “target protein” are used herein for expediency, but should not be considered limiting, unless specifically indicated, since the apparatuses and methods of the present invention equally permit purification of non-protein components.

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

The term “diatomaceous earth” or “DE” or “diatomite” or “kieselgu(h)r” is defined below and generally means any of a number of clay-based materials derived from the amorphous silica (opal, SiO₂·nH₂O) fossilized remains of aquatic organisms called diatoms (microscopic single-celled algae) found in lacustrine or marine sediments. Thus, DE is largely (80-90%) siliceous material from sedimentary rock crumbled into a fine white to off-white powder. Particle size of DE ranges typically from 10 to 200 μm. DE also contains trace minerals such as 2-4% alumina and 0.5-2.0% iron oxide. Other trace minerals commonly found in DE in various percentages include calcium, magnesium, copper, manganese, chromium, potassium, phosphate, copper, zinc, antimony, and the like. DE is commercially available under various trademarked names and trade names such as Celite® and Celpure®. DE is often calcinated and/or acid washed to provide additional functionality. DE is often provided commercially in the form of a powder and is easily measured and meted out in solution by forming a slurry with appropriate or compatible buffer and/or water.

As used herein, the term “resin” means a chromatography bead-based resin and encompasses both affinity resins and other chromatography resins commonly employed in biochemical fields for the purification and separation of complex mixtures.

A biological sample, as used herein, means a liquid sample, or lyophilized or powder sample that is solubilized, comprising biological components including, but not limited to, whole cells, virus particles, lysed cells, cell components including nucleus proteins, organelles, and the like. Biological samples are obtained from eukaryotes and prokaryotes, such as mammalian cells, insect cells, yeast cells, bacterial cells, and the like. Mammalian cells include, for example, human cells, rodent cells, bovine cells, equine cells, feline cells, canine cells, primate cells, and the like. Biological samples are either clarified or unclarified, meaning they are either substantially free of membrane components or not free of membrane components, respectively. Biological samples purified by the apparatus and methods disclosed herein optionally include a recombinant protein that is a target protein to be purified away from other cellular matter.

### Apparatus for Purification of Biological Samples

Purification of analytes away from interfering components in a biological sample is a critical component of biochemical research and development. Isolation of a target analyte for study is often required to characterize the function and structure of many biological components, including proteins, enzymes, cofactors, organelles, viral particles, capsids, just as an example. Single cell organisms and multi-cell organisms are comprised of complex arrangements of biological components and their study constantly requires developing new, efficient, and simple tools by which to isolate these components from surrounding milieu. For instance, the study of infectious agents, such as bacteria, viruses, viroids, fungi, protozoa, helminth, amoeba, algae, prions, metazoa, mycoplasma, all require that various components of these agents be grown, cultured, amplified, and then various biochemical sub-components isolated for study.

While much technology currently exists for separation and purification of biochemical components of biological samples, further improvement is always needed as new discoveries are made and new biochemical components are identified. Further improvement of existing technologies is also a necessity to keep pace with modern investigative science. To this end, purification techniques that are faster, more efficient, less costly, less wasteful, cleaner, and automatable or high throughput are constantly in need in the field of biochemical science and molecular biology, virology, parasitology, and similar fields of investigation.

Presented herein is a purification apparatus that achieves these goals. The apparatus is light, making it easy to ship, stable at various temperatures, simple in use, generates little waste, and is easily adapted to automation processes. Further, the apparatus melds well with existing chromatographic separation techniques currently available to researchers.

It has been surprisingly found that a purification media comprising a combination of a resin and diatomaceous earth (DE), supported by a membrane, creates a very functional purification apparatus with unexpected results. These surprising findings are at least twofold. Firstly, it has been found that by adding DE to chromatography resins, very dirty, crude, and even entirely unpurified or unclarified samples can be used to isolate components (i.e. analytes), e.g. proteins, of interest therefrom. Previously, additional steps were required to handle the sample, process it, remove contaminating material from it by centrifugation, chromatography steps, precipitation, and the like prior to isolation of the protein of interest such that the contaminating material would not interfere with the interaction between the protein of interest, or analyte, and the resin. However, it has now been discovered that by simply adding an amount of DE to the resin, these contaminating components no longer pose a threat to isolation of the analyte in mixed biological samples.

Previously, DE has been known to be used to form a “cake” or paste through which biological samples may be eluted to trap or capture contaminating components such as cell debris, organelles, vacuoles, vesicles, lysosomes, endoplasmic reticulum components, membrane components, and the like. However, disclosed herein is proof that, for the first time, it is found that DE can be incorporated into, mixed with, and bound to the resin and achieve remarkable results as outlined in the examples below. That is, DE in the present apparatus is in some embodiments mixed thoroughly with the resin and in this state is capable of purifying analytes from complex biological sample mixtures without any additional clarification of the sample prior to purification. While not wishing to be bound any specific theory, it is possible that the DE acts as a permeable filter cake, binding to cell components and contaminants, removing them from the liquid stream passing through the resin, and preventing these contaminants from clogging the resin or clogging the bottom membrane. This keeps the pores of the membrane open for non-specific proteins to pass through the purification media and allows the analyte or protein of interest to bind to or otherwise interact normally with the resin of choice.

Second, in addition to this surprising finding, it has been discovered and described herein that the purification media comprised of these components, resin and DE, can be thoroughly dried either as a mixture or as separate components, in a housing without loss of this remarkable function. It is clear that a dried purification apparatus weighs less than one comprising hydrated resins and gels and other components. What may not be clear is that even in this dried state, which is easier and cheaper to ship, the purification media described herein is relatively stable. Further, without the presence of liquid, there is no fear of loss of function or activity in the purification media at very low or freezing temps, conditions that could normally freeze resin and DE solutions causing the resin beads to become malformed and/or loose function. Thus, the dried apparatus comprising dry purification media affords numerous benefits not previously recognized in prior art media.

Finally, it has been discovered that even upon reconstitution of the dry purification media with an impure, unclarified, complex biological sample, and thoroughly mixing that sample with the DE and resin combination, the analyte or target protein is easily purified in a very quick succession of steps involving a wash and an elution through the membrane. The ease, efficiency, and simplicity of the described apparatus make it particularly amenable to automation and high throughput adaptations. These and other features of the purification apparatus are described in more detail below.

### Housing

The apparatus includes a housing that serves the purpose of holding purification media. The purification media acts as a chromatography column but is also useful for batch processes. The housing is generally not limited to any specific embodiment but instead can be adapted to any particular use, size, shape, or design, so long as it serves the function of holding the purification media and the sample such that they are allowed to mix, interact, and incubate, prior to being vacated from the housing. Thus, the housing comprises at least a top opening through which the biological sample is added, and a bottom opening, through which the purified material is obtained.

The housing is made of any polymer that does not interact with and does not stick to the component that is to be purified. The housing should also serve a third function, which is to allow sufficient mixing and interaction with the biological sample prior to washing and elution steps. Thus, the housing is, in one embodiment, made of silica glass or other doped-glass composite, plastic polymer, rubber and/or elastomeric compounds, and/or resin, and similar chromatography material. The housing is optionally biodegradable and comprised of, for instance, polyhydroxyalkanoates (PHAs), polylactic acid, starch blends, cellulosic-based plastics such as cellulose esters (cellulose acetate and nitrocellulose), celluloid, polyethylene terephthalate, polyethylene, polypropylene, polystyrene, polyglycolic acid, polybutylene succinate, polycaprolactone, poly(vinyl alcohol), polybutylene adipate terephthalate, and the like known biodegradable polymers. The housing is optionally comprised of metal, pure metal, or a metal alloy. The plastic polymer is in one embodiment selected from one or more of polycarbonate, polyetherimide, polyphenylsulfone, polystyrene, polysulfones, and polymethylpentene (also known as NALGENE®).

The housing is typically a round tube shaped as a column with a narrow tip at the end for collecting eluate. However, in another embodiment, the housing is a well in a microtiter plate similarly possessing an opening on both ends such that at one end sample and purification media can be added and the eluate can be collected from the opposite end. Such plates are often called “filter plates” in the art. In one embodiment, the housing is a well in a 96-well microtiter plate. In another embodiment, the microtiter plate is a 8, 24, 384, or 1536 well microtiter plate. Any number of such arrangements are commercially available and adaptable to the described purification apparatus. Particularly, for automation, 24-well microtiter plates are commonly used and easily adaptable to serve as housing for the described purification apparatus. Alternatively, the housing is of any particular geometric shape, such as a square, oval, ellipse, or the like, or a polygon. The housing has one opening for adding sample and purification media, and a second opening for liquid waste, washes, flowthrough, and eluate, to pass through while holding the purification media in place, as in a column chromatography operation. In one embodiment the housing is a spin column. In another embodiment, the housing is a single-use gravity flow column.

The housing is of any size amenable to biological purification processes; however, in certain embodiments the housing holds a volume of liquid equal to about 100 mL or less. In another embodiment, the housing holds a liquid volume of 90, 80, 70, 60, 50, 40, 30, 20, or even 10 mL or less. In a particular embodiment, the housing holds from 0.6 to 2.0 mL of liquid. In a further embodiment, the housing holds from 0.1 to 0.5 mL of liquid, or from 0.01 to 0.5 mL of liquid, or from 0.01 to 0.1 mL of liquid.

The housing holds the purification media and possesses sufficient volume to allow thorough mixing and interaction between the biological sample components and the purification media. Such mixing is in some embodiments aided by agitation, shaking, rocking, inverting, tilting, swinging, or other mechanical means of generating mixing between the biological sample and the purification media.

In one embodiment, the housing is adaptable to centrifugation, meaning that the housing, when subjected to centrifugal forces, maintains structural integrity. In some embodiments, the housing is sufficiently rigid to allow centrifugation up to speeds of 10,000×g without substantial loss of structural integrity, i.e. without loss of the sample or the purification media. In other embodiments, the housing is sufficiently rigid to sustain without substantial structural change centrifugal forces of up to 9,000×g, 8,000×g, 7,000×g, 6,000×g, 5,000×g, 4,000×g, 3,000×g, 2,000×g, 1,000×g, and 500×g. In a particular embodiment, the housing withstands centrifugal forces of at least 2,000×g without significant structural damage.

### Purification Media—Membrane

The housing comprises a membrane which supports the purification media, i.e. is below the purification media. The membrane is in one embodiment polytetrafluoroethylene (PTFE). The membrane is in some embodiments comprised of, either partially or in whole, a PTFE alternative such as, but not limited to, polychlorotrifluoroethylene (CTFE), perfluoroalkoxy (PFA), tetrafluoroethylene-perfluoropropylene, fluorinated ethylene propylene, polyether ether ketone (PEEK), ethylene tetrafluoroethylene (ETFE), polyvinylidene fluoride (PVDF), or various combinations thereof. In one embodiment, the membrane is made of 100% PTFE. In another embodiment, the PTFE membrane is a hydrophilic PTFE membrane compatible for use with aqueous, acidic, basic, non-aggressive organic, and aggressive organic solutions. In another embodiment, the membrane has a pore size of between 0.1 μm and 30 μm. In a further embodiment, the PTFE membrane has a pore size of between 0.5 μm and 20 μm, 1.0 μm and 15 μm, 1.5 μm and 10 μm, or between about 0.2 μm and about 0.45 μm, or between about 15 and 25 μm, or has an average pore size of about 20 μm. In another embodiment, the PTFE membrane is hydrophobic. While other membranes, such as nylon, PVDF, polyethersulfone (PES), cellulose acetate, polypropylene (PP), regenerated cellulose (RC), nitrocellulose (NC), and the like are substitutable for PTFE, and/or combinable with PTFE, in particular embodiments the membrane is 100% PTFE.

The amount of membrane material in the housing is not particularly limited so long as it satisfies the structural requirement of holding the purification media components in the housing during operation and does not markedly or detectable interact with the biological sample or analyte. In one embodiment, the membrane is essentially a disc covering the bottom of the housing just above the bottom opening. The housing and membrane are sufficiently rigid to withstand a vacuum pressure of about −0.8 bar. In another embodiment, the housing and membrane are sufficiently rigid to withstand without marked loss of function or structure of about −0.5 bar to about −1.0 bar.

### Purification Media

Purification media is added to the housing. The volume of purification media added to the housing depends on the particular application the operator has in mind, i.e. the type of separation or purification operation to be performed. Since the apparatus is adaptable numerous different types of purification media, the volume of purification media likewise varies depending on application. However, the housing must be sufficiently large to hold both the purification media and the sample to be purified. That being said, the sample is, in some embodiments, able to be split into 2, 3, 4, 5, 6, 7, or even 10 separate aliquots and processed in sequence using the same housing and purification media. In another embodiment, the housing and purification media are disposable after just the first use.

The purification media is comprised of at least two components, those being: 1) a resin, and 2) diatomaceous earth.

### Purification Media—Resin

The resin component of the purification media is in some embodiments an affinity resin. In some embodiments the affinity resin is selected from one or more of Protein A, Protein G, Protein L, heparin, and lectin. Affinity resins are those known in the art, such as Protein A, Protein G, Protein L, and immobilized metal affinity chromatography (IMAC) resins. IMAC resins commonly employ metal ions, such as Zn²⁺, Cu²⁺, Cd²⁺, Hg²⁺, Co²⁺, Ni²⁺, and Fe²⁺, that are bound to the resin to aid in interacting with affinity tags on targets of interest, such as a poly-histidine tag interaction with nickel, etc. Affinity resins also includes other ligand-protein type resins such as lectin resins, heparin resins, boronate resins, and any resin comprising an antibody or protein-based binding site with affinity for an epitope, such as but not limited to antibodies, ligands, receptors, fusion proteins, subunits of proteins, recombinant proteins, and fragments of the same. In a further embodiment the affinity resin comprises an antibody or antigen binding fragment thereof, such as, for example, an single chain variable fragment (scFv), antigen-binding fragment (Fab), variable region fragment (Fv), F(ab)2 fragment, minibody, diabody, triabody, tetrabody, bis-scFv, or other known functioning antibody fragment, and combinations thereof, coupled to a resin support. Any known antibody or antigen binding fragment thereof that is amenable to coupling to resin while maintaining antigen binding activity can be used as the affinity resin. Other non-affinity resins are those known in the art and include, for instance, ion exchange resins, size exclusion chromatography resins, hydrophobic interaction chromatography (HIC) resins, gel filtration, and the like. All of these resins are useful and employable in the context of the presently described apparatus purification media. In a particular embodiment, the resin is an affinity resin. In another embodiment, the resin is an IMAC resin. Particularly, in one embodiment the resin is a nickel-based IMAC resin. In another specific embodiment, the resin is a Protein A and/or Protein G resin.

### Purification Media—Diatomaceous Earth (DE)

The diatomaceous earth component of the purification media is one that is known in the art and commercially available under various trade names such as Celite® or Celpure®, which includes Celpure® 300, Celpure® 25, Celpure® 65, Celpure® 100, and Celpure® 1000 (sometimes sold with an additional letter “P” preceding the number, Imerys Filtration Minerals, Inc., San Jose, Calif., US). In a particular embodiment, the DE is Celpure® 300. As noted above, DE comprises in some embodiments various trace minerals. In one embodiment, the DE comprises: a) about 84.1 mg/kg aluminum, about 52.5 mg/kg calcium, about 50.5 mg/kg magnesium, about 20.0 mg/kg iron, about 10.5 mg/kg zinc, about 0.8 mg/kg copper, about 0.6 mg/kg antimony, about 0.7 mg/kg manganese, and about 0.2 mg/kg chromium; or b) about 6.2 mg/kg magnesium, about 2.8 mg/kg iron, about 0.6 mg/kg copper, and about 0.2 mg/kg manganese; or c) the following components:

| | % |
|---|---|
| SiO₂ | 98.65 |
| Al₂O₃ | 0.80 |
| Fe₂O₃ | 0.27 |
| Na₂O | 0.14 |
| K₂O | 0.10 |
| MgO | 0.08 |
| CaO | 0.08 |
| TiO₂ | 0.03 |
| P₂O₈ | 0.03 |

The DE used in the described purification media is in some embodiments calcinated and/or acid washed. Calcination and acid wash procedures are known in the art, but generally involve the steps of washing the DE with water, heating the washed DE to about 1000° C. to calcinate the DE, washing the calcinated DE in acid to create acid-washed DE, and heating the acid-washed DE in water to 200° C. to create dried DE.

### Purification Media—Relative Amounts of DE and Resin

The preferred amount of DE included in the purification media depends on the amount of sample to be added to the housing for purification. When the biological sample comprises mammalian cells, generally the amount of DE included in the purification media is from approximately 1 mg DE per 6.25×10⁴ mammalian cells to about 1 mg DE per 1.25×10⁶ mammalian cells, preferably from about 1 mg DE per 24×10⁴ mammalian cells to about 1 mg DE per 37.5×10⁴ mammalian cells.

When the biological sample comprises bacterial cells, the bacterial cell is preferably lysed prior to applying it to the top of opening of the housing, and the DE is present in an amount of less than approximately 1 mg DE per 2.5×10⁶ lysed cells.

The amount of DE present in the purification media is also definable by the amount of resin present in the purification media. Thus, about 24 mg of DE is present in the purification media per 50 μL of dried resin. In other words, said differently, in one embodiment, there is approximately 0.48 mg DE per μL of dried resin present in the purification media. In another embodiment, the amount of DE is present is from about 0.1 to about 0.9 mg DE per μL of dried resin, or from about 0.2 to about 0.8, from about 0.3 to about 0.7, from about 0.4 to about 0.6, or from about 0.4 to about 0.5 mg DE per μL of dried resin.

The purification media is present in the housing in a substantially dried state. It is well known that resins, especially affinity resins, are often shipped in a fully hydrated state, as a liquid mixture, often comprising various preservatives and additives. However, surprisingly, and advantageously, it has been discovered that the purification media described herein needs no liquid to be stable. Thus, the housing and purification media comprising the described apparatus is able to be shipped to end users in substantially dry form, with no liquid present in or around the housing or in or around the purification media. Clearly, shipping dry material such as the described apparatus has numerous advantages, especially if the apparatus is functionally stable. Shipment of a dry apparatus costs far less than shipping liquid compositions. Further, a dry apparatus such as described herein is more easily transportable to remote areas where on-site testing may be desired for the presence of one or more infectious diseases or causative agents. Additionally, stability in the dry state provides numerous advantages in terms of on-site testing in extreme heat or extreme cold where there is no fear of freezing and perhaps incurring damage to the apparatus due to freezing of liquids. Particularly, it is noted that this apparatus in its dry state is stable for at least 30 days, 40 days, 50 days, 60 days, 90 days, 120 days, or 360 days or more at room temperature and/or at 4° C., making its shipment and use in various geographic regions possible without specialized care and handling. The term “stability” as used herein is meant to refer to the maintenance, or avoidance of loss of binding capacity over time and under various different conditions.

Optionally, in some embodiments, the purification media will include various known preservatives and/or additives, as mentioned above. Additionally, or alternatively, in some instances, for example, a wetting agent is included with the purification media or resin. Wetting agents and surface modifiers are interfacially active substances that enhance different component capabilities as they relate to coating functionality. A wetting agent is an additive that increases the spreading and penetrating power of an aqueous liquid on a solid substrate. The wetting agent is in some instances an emulsifier, emollient (humectant), and/or a surfactant. For instance, standard non-ionic surfactants are in some instances employed as the wetting agent and have an hydrophilic-lipophilic balance (HLB) value of between 7 and 15.

It is noted that especially in embodiments that include, for instance, a proteinaceous component, such as in embodiments incorporating protein A or protein G in the purification media, addition of a wetting agent assists in maintaining stability, and therefore functionality, i.e. binding capacity, of the apparatus. Thus, in some embodiments such wetting agents are added to increase stability and shelf life of the apparatus. Without wishing to be bound by any specific theory, it is postulated that the wetting agent added to such embodiments helps to maintain moisture around the protein affinity components of such purification resins so that the protein affinity components maintain their binding functionality throughout the drying process. A suitable wetting agent for this purpose is, for instance glycerol, and like compounds known in the art typically employed for such purposes.

Examples of non-surfactant wetting agents are water soluble solvents and alkali metal hydroxides. Non-limiting exemplars of wetting agents include anionic, cationic, nonionic, and amphoteric wetting agents. Anionic, cationic, and amphoteric wetting agents ionize when mixed with water. Wetting agents include compounds such as nonionic and ionic surfactants, such as glycerol, alkoxylated surfactants, silicone surfactants, sulfosuccinates, fluorinated polyacrylates, and star-shaped polymers (a class of alkoxylated surfactants).

Suitable ionic surfactants are, for example, alkali metal and alkaline earth metal salts of alkylarylsulfonic acids having a straight-chain or branched alkyl chain, such as phenylsulfonate CA or phenylsulfonate CAL (Clariant), ®Atlox 3377BM (Uniqema), ®Empiphos TM series (Huntsman); polyelectrolytes, such as lignosulfonates, polystyrenesulfonate or sulfonated unsaturated or aromatic polymers (polystyrenes, polybutadienes or polyterpenes), such as ®Tamol series (BASF), ®Morwet D425 (Witco), ®Kraftsperse series (Westvaco), ®Borresperse series (Borregard). Suitable nonionic surfactants are, for example, polyalkoxylated, preferably polyethoxylated hydroxy fatty acids or glycerides containing hydroxy fatty acids, such as, for example, ricinine or castor oil having a degree of ethoxylation between 10 and 80, preferably from 25 to 40, such as, for example, ®Emulsogen EL series (Clamant) or ®Agnique CSO series (Cognis). Suitable surfactants on a nonaromatic basis are, for example, fatty acid amide alkoxylates, such as the ®Comperlan products from Henkel or the ®Amam products from Rhodia; alkylene oxide adducts of alkynediols, such as the ®Surfynol products from Air Products. Sugar derivatives, such as amino sugars and amido sugars from Clariant, glucitols from Clariant, alkyl polyglycosides in the form of the ®APG products from Henkel or such as sorbitan esters in the form of the ®Span or ®Tween products from Uniquema or cyclodextrin esters or cyclodextrin ethers from Wacker; surface-active polyacryl and polymethacryl derivatives, such as the ®Sokalan products from BASF. (See, for instance, U.S. Pat. App. Pub. No. 2008/0318774, incorporated herein by reference for all purposes).

In some embodiments the wetting agent is one or more of alkali metal hydroxides or water soluble C₃-C₆ alcohols or polyols, glycols, and glycol ethers, such as water soluble C₃-C₆ primary alcohols. Non-limiting examples include sugar alcohols, such as, for instance, sorbitol, mannitol, erythritol, xylitol, lactitol, isomalt, maltitol, and the like. Suitable alkali metal hydroxides include sodium hydroxide, potassium hydroxide, lithium hydroxide, rubidium hydroxide and cesium hydroxide. Sodium hydroxide and potassium hydroxide are preferred for cost reasons. Alkali metal hydroxides can suitably be applied in concentrations from 0.1-10 wt %, such as 2-8 wt %. Non-limiting examples of water soluble C₃-C₆ alcohols are n-propanol, isopropanol, n-butanol, isobutanol, 2-butanol, tert-butanol, pentanols, hexanols and benzyl alcohol, of which n-propanol, n-butanol, n-pentanol, n-hexanol and benzyl alcohol. N-propanol and isopropanol are completely water-miscible at room temperature. Examples of water soluble (and completely water-miscible) C₃-C₆ glycols include 1,2-propanediol, 1,2-butanediol, 1,2-pentanediol, 1,2- hexanediol, 1,3-propanediol, 1,4-butanediol, diethylene glycol, and the like. Examples of water soluble (and completely water-miscible) C₃-C₆ glycol ethers include ethylene glycol methyl ether, ethylene glycol ethyl ether, ethylene glycol propyl ether, ethylene glycol butyl ether, diethylene glycol methyl ether, diethylene glycol ethyl ether, propylene glycol methyl ether, propylene glycol ethyl ether etc. In one embodiment the wetting agent comprises 0.1-30 wt %, such as 2-30 wt %, 3-20 wt % or 4-10 wt % of one or more C₃-C₆ alcohols, polyols, glycols or glycol ethers.

In one particular embodiment, the wetting agent is included at from 0.1 to 5 wt % and is glycerol. In another specific embodiment, the glycerol is included at about 5 wt %, or from about 4 wt % to about 6 wt %. In some instance the amount of wetting agent is between 10 wt % and 30 wt %, or as high as 20 wt %.

### Biological Samples

As noted above, the biological sample may be any sample comprising biological material, i.e. biological molecules. The biological sample may comprise target protein, contaminating proteins, cells, whole cells, lysed cells, cell components and/or organelles, cell membrane structures, enzymes, cofactors, viral particles, and/or capsids, just as examples. The biological sample comprises, in some embodiments, a mixture of one or more of bacteria, viruses, viroids, fungi, protozoa, helminth, amoeba, algae, prions, transposons, metazoa, and mycoplasma, and components thereof, or other known infectious disease or causative agent of infection. Preferably, the sample comprises cells. Preferably, the cells are eukaryotic or prokaryotic cells. Preferably the cells are selected from, for example, yeast cells, bacterial cells, mammalian cells, plant cells, insect cells, human cells, and the like. Preferably the cells are mammalian cells. Preferably the sample is a sample obtained from a subject or patient, preferably a human subject or patient, and may be a bodily fluid or tissue homogenate of the patient. Biological samples are in the form of a liquid, most often, but are also in some embodiments a dried sample or lyophilized sample that is later hydrated before purification. In another embodiment, the sample is a water sample, or soil sample. In one embodiment, the biological sample comprises an expressed recombinant protein. In another embodiment, the expressed recombinant protein is secreted outside cells in the biological sample. In another embodiment, the protein is not recombinant and is secreted. In a further embodiment, the target analyte is not secreted and cells in the biological sample are lysed in order to gain access to the analyte inside the cell.

In a further embodiment, the analyte is an expressed recombinant protein engineered with an affinity tag. In some embodiments, the affinity tag is an immunoglobulin domain or a histidine tag or another antigen recognized by an antigen binding fragment linked to the affinity resin. In some embodiments the expressed recombinant protein comprises a FLAG affinity tag or a GFP affinity tag. Any known affinity tag can be added to the recombinant protein for the purpose of purification so long as a known antigen binding domain is available to link to the affinity resin by known methods. In a particular embodiment, the expressed recombinant protein comprises a histidine tag that complexes with cobalt or nickel.

### Methods of Manufacturing and Uses of the Purification Apparatus

Provided herein are methods of manufacturing or preparing the described purification apparatus, as well as downstream uses thereof. While housing can be obtained commercially, the relative amounts of the purification media components and their make-up, as well as their mode of assembly, are further defined below.

### Methods of Manufacture

In some embodiments, the housing is acquired commercially and already contains within it the requisite volume capacity, along with a top opening, a bottom opening, and a membrane positioned between said top opening and said bottom opening. As mentioned above, typically the membrane is closer to the bottom opening than the top opening of the housing. Methods of manufacturing such housings are known in the art and adaptable to the presently described purification apparatus.

Other components of the purification media are then added to the housing. Purification media additionally comprises resin and DE. Preferably, initially, the resin is suspended in an aqueous liquid media to create a slurry of hydrated resin. An amount of hydrated resin is then added to the housing such that it directly contacts the membrane. The slurry is, in one embodiment, a 50:50 slurry of resin to solution. However, in other embodiments, the slurry is any other ratio of resin to solution that is amenable to pipetting or otherwise allowing reproducible transfer of the resin to the housing. The solution is in one embodiment a buffered solution compatible with the resin, i.e. that does not cause rapid degradation or loss of function to the resin. The aqueous solution in some embodiments comprises water, an alcohol (such as ethanol), and optionally a preservative.

In addition to resin, DE is added to the housing. The DE is, in one embodiment, added also as a slurry of DE suspended in a solution. In one embodiment, the slurry is a 50:50 slurry of DE to solution. The solution is in one embodiment a buffered solution compatible with the DE, i.e. that does not cause rapid degradation or loss of function to the DE, or water.

In another embodiment, the DE is first added followed by resin. In a further embodiment, the DE and resin are thoroughly mixed while in liquid form. Mixing is achieved by any known means such as tilting, inverting, shaking, vibrating, vortexing, and the like, or any mechanical agitation suitable and compatible with the resin, housing, membrane, and other components. In a particular embodiment, the resin and DE solutions are not substantially mixed but instead layered one atop the other thereby creating an interface between the upper layer and lower layer of the purification media. In another embodiment, some mixing occurs at the interface between the upper layer and lower layer.

The housing comprising the membrane, resin, and DE is then dried until it is substantially free from liquid. Drying is accomplished by exposure to air at ambient temperature, heating to a temperature that does not cause degradation to the housing or purification media, or other apparatus components such that structure integrity and/or function is not diminished.

In another embodiment, the DE and resin are added as dry powders to the housing. In a further embodiment, the housing is a microtiter plate and upon addition of the purification media and the drying step, a seal is added to the top of the plate to protect the purification media components from loss, the addition of any contamination, or from exiting the housing through the top opening, in shipping prior to use. In one embodiment the seal is a ClearVue seal or the like (Molecular Dimensions, Maumee, Ohio, US). In another embodiment the seal or cap is paraffin, wax, a plastic shrinkwrap or other thin plastic sheet capable of fixedly attaching to the top opening of the housing.

### Methods of Use of the Purification Apparatus

The aim of the apparatus is to provide a simple, efficient, reproducible, stable, and environmentally friendly purification apparatus to isolate and/or purify a desired component (analyte), preferably a protein, from a biological sample. In such a method, a first step is to add the biological sample to the apparatus.

If the biological sample is in dry form as a lyophilized powder or freeze-dried sample, the biological sample is hydrated with an appropriate solution that is compatible with the purification media, either before or after addition to the apparatus. When the biological sample is added to the housing, the purification media becomes rehydrated.

In one embodiment, the biological sample and purification media are agitated by use of a mechanical electrically-powered motion device to promote thorough mixing. It is desirable to ensure that the biological sample components have optimal opportunity to contact and bind to the resin and interact with the DE components of the purification apparatus. To achieve this goal, various forms of agitate are employed as known in the art, such as vortexing, shaking, tilting, inverting, and the like by an electrical machine with a motor that is preferably programmable and provides a secure surface upon which to attach the housing without loss of biological sample or purification media. The mixing is in one embodiment conducted at room temperature or ambient temperature. In other embodiments, where the analyte is temperature sensitive, perhaps due to the presence of proteases in the biological sample, the mixing is performed at cooler temperatures, such as at 10° C., 8° C., 4° C., or 0° C. When tilting or inverting the apparatus, in some embodiments it is necessary also to cap the apparatus to avoid loss of biological sample. In an optional embodiment, the apparatus comprises such a cap that both air-tight and water-tight.

After mixing, the apparatus is preferably cleared of liquid to yield what is termed the “flowthrough.” The liquid may be cleared from the apparatus either by gravity flow, by centrifugation, or application of a vacuum. In one embodiment, the vacuum pressure applied to the apparatus is between about −0.5 bar and −0.8 bar. In another embodiment, the apparatus is centrifuged at about 2,000 to 3,000×g.

After liquid is removed from the apparatus, the analyte is preferably removed from the apparatus. Optionally, the purification media is first washed one or more times with a compatible buffer to remove non-binding components. However, care should be taken to avoid performing so many wash steps that analyte is lost in the washing steps and not recovered. Thus, there is a balance between the number of washing steps to remove extraneous contaminants that do not bind the resin, and the loss of analyte with each wash and the skilled person is readily able to determine the number of wash steps appropriate for his/her purposes.

Finally, an elution buffer is preferably applied to the purification apparatus. Elution buffers are well-known in the field and any suitable elution buffer may be used in the methods of the present invention. An elution buffer typically comprises components specially designed to mask the binding sites of the resin or otherwise interfere with binding of the analyte to the resin. The elution buffer components therefore typically depend directly on the type of resin employed for the purification protocol. In one embodiment, the resin is an IMAC resin and the elution buffer comprises a buffering agent, a salt, and imidazole at concentrations known to disrupt the interacting of the ligand with the metal. In another embodiment, the resin is an affinity resin, such as Protein A, Protein G, or Protein L, and the elution buffer comprises a buffering agent, glycine HC1, and has a pH that is between 2.5 and 3.0 to disrupt any antibody-antigen interactions on the resin.

After removal of the analyte from the purification apparatus, the purification apparatus is in one embodiment discarded. In another embodiment, the purification apparatus is reused by first regenerating the resin, then washing the purification media, and then drying the purification media as shown above. In one embodiment, the housing comprises biodegradable plastic polymers such that upon being discarded the impact of the empty housing on the environment is minimized.

In one embodiment, the collected eluate is then analyzed for the presence of the analyte. Such analytical and quantitative methods of detection are well known in the art and include, for instance, SDS-PAGE, NMR, HPLC, ELISA, PCR, further chromatography steps, FPLC, immunoprecipitation, CD, protein crystallography, dotblot, Western blot, Eastern blot, and the like.

### Systems Comprising the Biological Purification Apparatus

Further contemplated herein are systems and automated systems for purifying biological samples that include incubators, shakers, liquid handlers, centrifuges, pipettes, vacuums, and other automated robotics incorporating the described purification apparatus.

Further modifications and alternative embodiments of various aspects of the methods and systems described herein will be apparent to those skilled in the art in view of this description. Accordingly, this description is to be construed as illustrative only and is for the purpose of teaching those skilled in the art the general manner of carrying out the disclosed methods and systems. It is to be understood that the forms of the disclosed methods and systems shown and described herein are to be taken as examples of embodiments. Elements and materials may be substituted for those illustrated and described herein, parts and processes may be reversed, and certain features of the disclosed methods and systems are capable of being utilized independently, all as would be apparent to one skilled in the art after having the benefit of this description of the disclosed methods and systems. Changes may be made in the elements described herein without departing from the spirit and scope of the disclosed methods and systems as described in the following claims.

All of the references cited above, as well as all references cited herein, are incorporated herein by reference in their entireties. The following examples are offered by way of illustration and not by way of limitation.

### EXAMPLES

### Example 1

### GFP Cross-Contamination Test

The biological purification apparatus was assembled as described above. In each instance, Green fluorescent protein (GFP) was added to an 96-well microtiter plate (Protein Ark, Ltd., Sheffield, UK). Recombinant GFP was expressed from a pEG-pET15B expression vector from BL21 (DE3) Escherichia coli cells induced with 1 mM IPTG. Bacteria were grown in LB or TB media at 37° C. for 8 hours or overnight, per manufacturer's standard protocols. Recombinant His-tagged GFP was recovered from cells that were pelleted and then lysed with BugBuster® from Millipore Sigma (St. Louis, Mo., US) using manufacturer's protocols, though any bacterial lysis buffer would be appropriate including lysozyme and benzonase and the like. The purification plate is commercially available prepackaged with polytetrafluoroethylene (PTFE) membranes.

To each well is then added 600, 800, or 1000 μL of a 1 mg/mL GFP solution in phosphate-buffered saline (PBS). Each neighboring well possessed an equal volume of PBS. The plates were then mounted onto a Stuart microtiter plate shaker. (Cole Palmer, Staffordshire, UK). Different shaker speeds were selected for testing (600 rpm, 1000 rpm, and 1250 rpm) and each microplate was shaken for approximately 15 minutes at ambient temperature. An aliquot of 50 μL was transferred to a 96-well Greiner Plate and GFP fluorescence quantitated by fluorimetry at 509 nm.

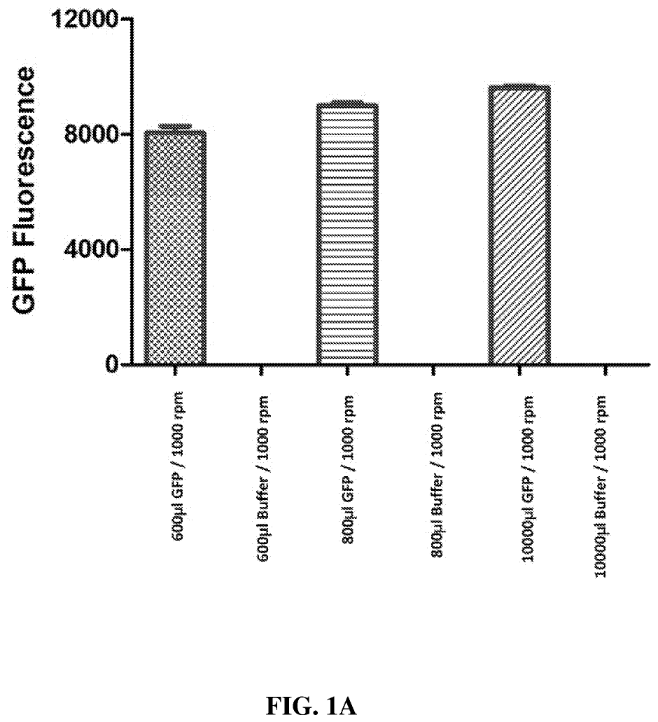

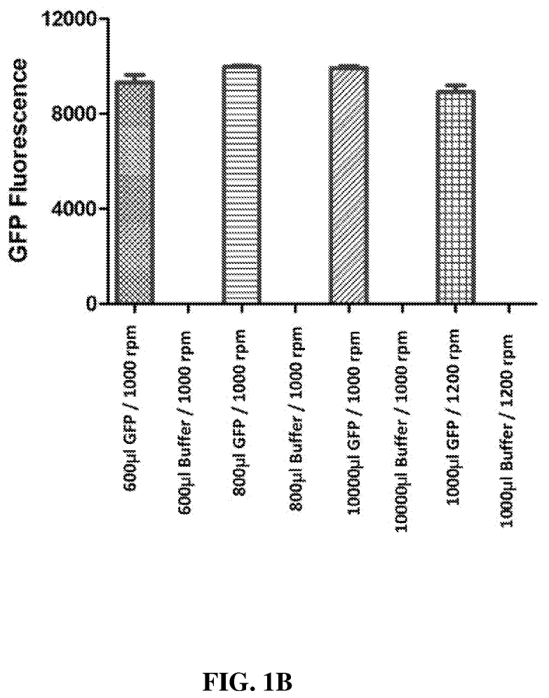

Results are shown in FIG. 1A and FIG. 1B, showing no detectable GFP found in any neighboring wells.

### Example 2

### Range of DE Amounts

In this experiment, the degree of clogging at high sample cell density was investigated with and without addition of DE. Here, mammalian cells were added to the purification apparatus prepared as in Example 1. The mammalian cells employed in this example are transformed human embryonic kidney (HEK 293 6E cells; see Cellosaurus accession number HEK293-EBNA1-6E (RRID:CVCL_HF20); U.S. Pat. No. 8,551,774; see also Jager et al., BMC Proceedings, 9:P40, 2015; Jager et al., BMC Biotechnol., 13:52, 2013; National Research Council (NRC), Canada) cells transiently expressing a secreted form of immunoglobulin G (IgG4, both heavy and light chains).

In this example, 100 μL of a 50:50 slurry of Protein A resin (Fastback Protein A Resin, Protein Ark, Ltd., Sheffield, UK; Protein A is Staphylococcal Protein A, 41 kDa, recombinant, from Prospec-Tany Technogene Ltd., Rehovot, Ill.) was added to an 96-well microtiter plate containing a PTFE membrane (Protein Ark, Ltd., Sheffield, UK). The Protein A resin is a Sepharose® Fast Flow resin (Protein Ark, Ltd., Sheffield, UK) with a ligand density of 3.5 mg Protein A per mL fully hydrated resin (as shipped), with bead size of 60 to 165 μm, and a binding capacity for human IgG antibody of about 30 mg/mL. The resin was resuspended in suspension buffer including distilled water with 0.01% thimerosal water and allowed to normalize at room temperature to create a 50:50 slurry. Then, 100 μL of the 50:50 resin slurry was pipetted into each well. The 96-well plate is commercially available prepackaged with polytetrafluoroethylene (PTFE) membranes at the bottom from Protein Ark, Ltd., Sheffield, UK.

The empty wells of the 96-well microtiter plates were loaded with 100 μL of a 50:50 Protein A slurry either with or without a further addition of 100 μL of 50:50 slurry of DE layered on top (Celpure® 300, Imerys Filtration Minerals, Inc., San Jose, Calif., US, about 24 mg DE per well). The 96-well microtiter plates were then allowed to dry at ambient temperature in a positive flow hood.

Generally, 600 μL of HEK cells at different cell densities were added to the wells along with the indicated amounts of DE in the same manner as described above with 50 μL dry Protein A resin, as above. Results of this experiment are provided in Table 1.

TABLE 1

| Cell density | DE | Volume | |
|---|---|---|---|
| (cells/mL) | (mg) | (ml) | Clogging |
| 10 × 10⁶ | 2.5 | 0.6 | Partial clogging |
| 10 × 10⁶ | 5 | 0.6 | All sample passed through |
| 10 × 10⁶ | 15 | 0.6 | All sample passed through |
| 10 × 10⁶ | 25 | 0.6 | All sample passed through |
| 50 × 10⁶ | 15 | 0.6 | Partial clogging |
| 50 × 10⁶ | 25 | 0.6 | All sample passed through |
| 50 × 10⁶ | 50 | 0.6 | All sample passed through |
| 50 × 10⁶ | 100 | 0.6 | All sample passed through |

At lower cell densities of 10×10⁶ cells/mL a lower amount of DE can be used without clogging the wells, i.e., 5 mg of DE is sufficient. For very high cell densities of 50×10⁶ cells/mL, 25 mg of DE is the lowest amount that can be used such that the entire sample still passes through the purification media without clogging. This experiment analyzed a range of cell densities per mg of DE including 2.4×10⁵ cells/mg DE to about 2.4×10⁶ cells/mg DE. The samples that exhibited partial clogging are estimated to be at the higher end of about 2×10⁶ and 2.4×10⁶ cells/mg DE, respectively (first sample and fifth sample in Table 1).

### Example 3

### Purification of His-Tagged ULP Protease from HEK Cells

This experiment is another type of control that is aimed at investigating whether a recombinant protein added to whole mammalian cells is able to be purified from whole cells. The mammalian cells employed in this example are transformed human embryonic kidney (HEK 293 6E cells; see Cellosaurus accession number HEK293-EBNA1-6E (RRID:CVCL_HF20); U.S. Pat. No. 8,551,774; see also Jager et al., BMC Proceedings, 9:P40, 2015; Jager et al., BMC Biotechnol., 13:52, 2013; National Research Council (NRC), Canada) cells transiently expressing a secreted form of immunoglobulin G (IgG4, both heavy and light chains). HEK cells were grown in F17 FreeStyle™ expression medium (ThermoFisher Scientific, Waltham, Mass., US) to confluency of 3×10⁶ cells/mL and collected. Cells were centrifuged and resuspended in F17 media at the indicated cell densities.

The purification apparatus employed in this example is again the 96-well microtiter plate as in Example 2, which was prepared just as in Example 2 except that the resin in this example is Fastback Nickel Advance resin. (Protein Ark, Ld., Sheffield, UK). The Fastback Nickel Advance resin is comprised of 6% cross-linked agarose and has a binding capacity per manufacturer of 80 mg/mL, a bead size of 90 μm, and a metal ion capacity of more than 75 μmol/mL. In this resin, nickel ions are loaded onto an agarose matrix via chelating coupled ligand to obtain a stable affinity matrix with a high binding capacity for histidine residues. The nickel resin is suspended in suspension buffer (distilled water with 20% ethanol) to create a 50:50 slurry. As in Example 2, 100 μL of the 50:50 slurry of resin is added to each housing on top of the PTFE membranes, followed by a 50:50 slurry of DE for some of the samples, as indicated below.

In this example the HEK cells are spiked at confluence and prior to collection with approximately 1 mg of His-Tagged ULP protease protein in 600 μL phosphate-buffered saline (PBS) at the range of cell densities tested in order to simulate a secreted His-tagged protein to be purified away from the cell samples.

To make the His-tagged ULP protease, the recombinant protein is expressed from pET28 expression vector in E. coli BL21(DE3) cells in Luria broth (LB) media and induced with IPTG per known protocols. The His-tagged ULP protease is purified from bacterial lysates with Fastback Nickel Advance Resin. (Protein Ark, Ld., Sheffield, UK) and polished by passing through size exclusion resin. Samples were incubated for 15 minutes at ambient temperature with shaking at 600 rpm on a Grant microplate shaker (Grant Instruments, Cambridgeshire, UK).

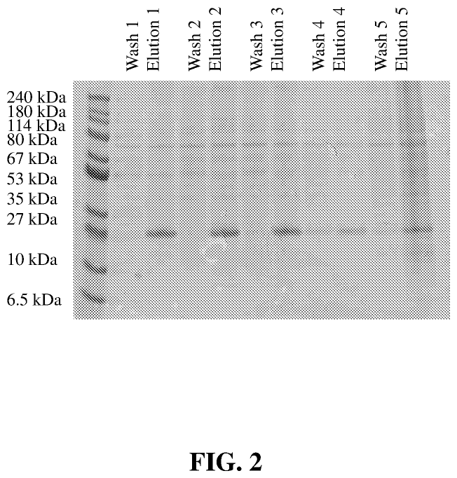

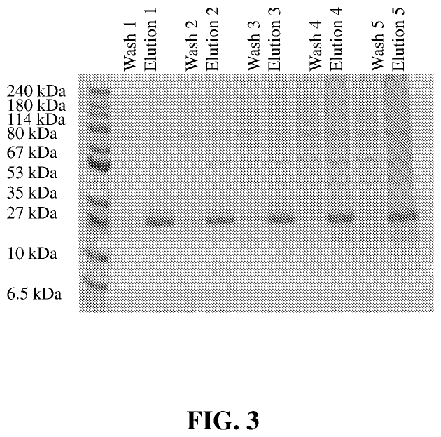

For purified sample recovery, whole cells were washed with 600 μL binding buffer (1×phosphate-buffered saline, PBS, containing 25 mM imidazole). Washes were repeated two times per plate. After the final wash, samples were eluted three times with 200 μL IMAC elution buffer (250 mM imidazole in PBS) and collected into a pellet by centrifugation. Wells were visually inspected for evidence of clogging and protein concentration at each step was detected by absorbance at 280 nm. Wash and eluate samples were analyzed by SDS-PAGE. (See, Table 2, below, and FIGS. 2 and 3).

For SDS-PAGE analysis 10 μL of each eluate was loaded onto 4-12% SDS-PAGE gels and electrophoresed for 1 hr at room temperature in 2-(N-morpholino)ethanesulfonic acid (MES) buffer.

FIGS. 2 and 3 provide images of stained SDS-PAGE gels for the same samples, with FIG. 2 representing samples purified without DE and FIG. 3 representing samples purified with DE. Sample 1 contained 2.5×10⁶ cells/mL. Sample 2 contained 5.0×10⁶ cells/mL. Sample 3 contained 10.0×10⁶ cells/mL. Sample 4 contained 25.0×10⁶ cells/mL. Sample 5 contained 50.0×10⁶ cells/mL. In Table 2, samples marked with a “+” sign indicates the presence of DE in those samples.

TABLE 2

| | Cell Density | DE | Post Elution | Protein Recovery |
|---|---|---|---|---|
| Sample | (cells/mL) | (mg) | Volume (μl) | (100% = 1 mg) |
| 1 | 2.5 × 10⁶ | 0 | 600 | 98 |
| 2 | 5.0 × 10⁶ | 0 | 600 | 96 |
| 3 | 10.0 × 10⁶ | 0 | 600 | 96 |
| 4 | 25.0 × 10⁶ | 0 | 600 | 50 |
| 5 | 50.0 × 10⁶ | 0 | 50* | 45 |
| 1+ | 2.5 × 10⁶ | 24 | 600 | 98 |
| 2+ | 5.0 × 10⁶ | 24 | 600 | 96 |
| 3+ | 10.0 × 10⁶ | 24 | 600 | 98 |
| 4+ | 25.0 × 10⁶ | 24 | 600 | 98 |
| 5+ | 50.0 × 10⁶ | 24 | 600 | 99 |

*Sample clogged

These cell densities (cells/mL) per 24 mg DE tested in this, and the following Examples, yield a cell/mg DE range of approximately from 6.25×10⁴ cells/mg DE to 1.25×10⁶ cells/mg DE. From Example 3, it is readily apparent from the results of the quantitative analysis based on protein absorption shown in Table 2 that presence of DE markedly improved recovery of target protein His-tagged ULP protease at higher cell densities. The recover improved surprisingly from 50 and 45% without DE to 98 and 99% with DE, doubling recovery.

### Example 4

### Purification of His-Tagged ULP Protease from Sf9 Insect Cells

This experiment is similar to the control of Example 3 and represents another type of control that is aimed at investigating whether a recombinant protein (His-tagged ULP protease, as in Example 3) added to whole mammalian cells is able to be purified from whole cells. The mammalian cells employed in this example are insect cells (Sf9, from Thermo Fisher Scientific, Waltham, Mass., US). The resin used in this Example was Fastback Nickel Advance Resin as in Example 3. (Protein Ark, Ld., Sheffield, UK). Generally, the same protocol was followed for generating the purification apparatus in that the same 96-well microtiter plate comprising the PTFE membrane was used as the base as in Examples 2 and 3, with addition of 100 μL of a 50% slurry of Fastback Nickel Advance resin followed by addition of 100 μL of a 50% slurry of DE as in Examples 2 and 3. However, in this example, the amount of DE in each apparatus was 25 mg. These plates were then dried in ambient temperature in a positive flow hood until substantially dry.

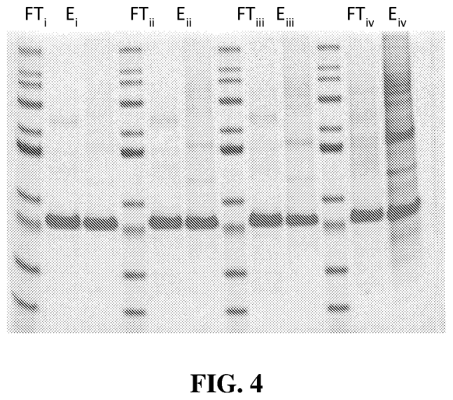

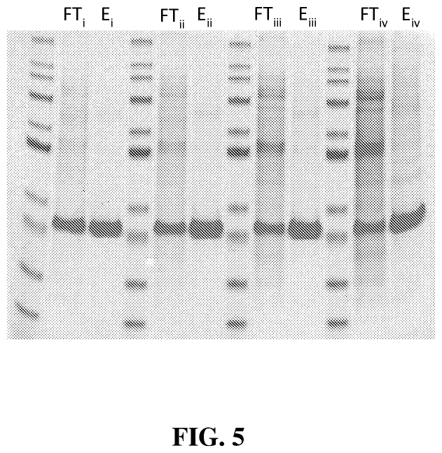

The Sf9 insect cells were grown in Sf-900 III media at 27 ° C. with shaking at 120 rpm, and grown to a cell viability of 96%. Various cell densities (2.5×10⁶ cells/ml, 5×10⁶ cells/mL, 10×10⁶ cells/mL, 15×10⁶ cells/mL, 25×10⁶ cells/mL, and 50×10⁶ cells/mL) were added to the purification apparatus, as whole cells (not lysed), with the addition of approximately 1 mg of His-tagged ULP protease (as in Example 4) and subjected to shaking in a Stuart microtiter plate shaker (Cole Palmer, Staffordshire, UK) for 15 minutes at 600 rpm. The purification apparatus was then washed three times with 600 μL wash buffer (as in Example 3) and then followed by two rounds of elution with 300 μL IMAC elution buffer. The plates were then spun at 2000×g for 2 minutes. Aliquots of each sample were assayed by SDS-PAGE as above. (See, FIGS. 4 and 5).

As shown in FIGS. 4 and 5, where “FT” means flowthrough (or wash), and “E” means eluate, very good purification of the recombinant His-tagged ULP was observed at every cell density up to sample iv where partial clogging of the column was observed. The cell densities of the various assayed samples included: i) 2.5×10⁶cells/mL, ii) 5.0×10⁶ cells/mL, iii) 10.0×10⁶ cells/mL, and iv) 25.0×10⁶ cells/mL. FIG. 4 contained no DE, whereas FIG. 5 contained 25 mg DE per well. It can be seen by visual inspection and comparison of FIGS. 4 and 5 that surprisingly, addition of DE markedly increased yield and purity of the recovered His-tagged ULP protease.

### Example 5

### Purification of His-Tagged GFP from E. Coli Cell Lysates

In this Example, the performance of the biological sample purification apparatus is tested with bacterial cells expressing a recombinant protein. The bacterial cells are Escherichia coli BL21(DE3) cells. (New England Biolabs, Ipswich, Mass., US). The protein expressed was His-tagged Green Fluorescent Protein (GFP) as in Example 1. Recombinant GFP was expressed from a pEG-pET15B expression vector induced with 1 mM IPTG. Bacteria were grown in LB or TB media at 37° C. for 8 hours or overnight, per manufacturer's standard protocols. Recombinant His-tagged GFP was recovered from cells that were pelleted and then lysed with BugBuster® from Millipore Sigma (St. Louis, Mo., US) using manufacturer's protocols, though any bacterial lysis buffer would be appropriate including lysozyme and benzonase and the like.

Samples were then vortexed to homogenise, and rocked for 20 minutes at room temperature. Samples were then sonicated for 3 bursts of 5 seconds each on ice. Approximately 600 μL of unclarified E. coli cell lysate was then added to Fastback Nickel Advance purification apparatuses as in Examples 3 and 4, with plates either containing DE or not containing DE prior to exposure to sample. Samples were shaken on a Grant microplate shaker (Grant Instruments, Cambridgeshire, UK) for 15 minutes. Samples were then centrifuged at 2000×g for 2 minutes at room temperature and washed three times with 600 μL binding buffer and eluted two times with 600 μL IMAC elution buffer as in Example 4. (See, Tables 3 and 4).

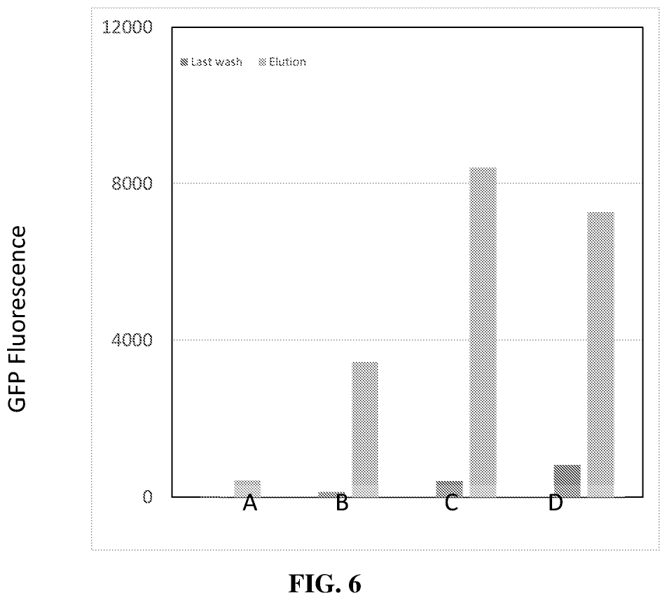

Results are shown in Tables 3 and 4, below, and FIG. 6. FIG. 6 provides a bar graph of GFP fluorescence measurement data from Samples A, B, C, and D, including GFP measurement from the last wash as well as from the elution (dark bar graph is GFP measured in the last wash and the lighter bars indicate GFP detected in the elution).

TABLE 3

| Sample | Cell pellet:Bug buster volume | | Remark |
|---|---|---|---|
| A | 0.1:6 | (10 mg + 590 μl) | Normal flow |
| B | 0.25:6 | (25 mg + 575 μl) | Normal Flow |
| C | 0.5:6 | (50 mg + 550 μl) | Normal Flow |
| D | 1.0:6 | (100 mg + 500 μl) | Partially clogged |
| E | 2.0:6 | (200 mg + 400 μl) | Clogged |
| F | 3.0:6 | (300 mg + 300 μI) | Clogged |

TABLE 4

| | | | | | Post Wash | Post Elution | |
|---|---|---|---|---|---|---|---|
| | Cell:Lysis | | Starting | | Volume (μl) | Volume (μl) | |
| | Buffer ratio | | Volume (μl) | Flowthrough | (500 μl | (200 μl | |
| | (g/mL) E. coli | | (mix @ | Volume (μl) | wash buffer | elution buffer) | |
| | Expressing | DE | 800 rpm, | (3000 × g, | (2000 × g, | (2000 × g, | |
| Sample | His-GFP | (mg) | 30 min) | 2 min) | 2 min × 2) | 2 min) | Clogging |
| A | 1:2 | 0 | 600 | 250 | 1000 | 200 | Partial at wash and elution |
| | (0.51/1.02) | | | | | | |
| B | 1:4 | 0 | 600 | 250 | 1000 | 200 | Partial at wash and elution |
| | (0.58/2.32) | | | | | | |
| C | 1:6 | 0 | 600 | 250 | 1000 | 200 | Partial at wash and elution |
| | (0.67/4.2) | | | | | | |
| D | 1:8 | 0 | 600 | 250 | 1000 | 200 | Partial at wash and elution |
| | (0.49/3.92) | | | | | | |
| A+ | 1:2 | 24 | 600 | 250 | 1000 | 200 | Partial at wash and elution |
| | (0.51/1.02) | | | | | | |
| B+ | 1:4 | 24 | 600 | 250 | 1000 | 200 | Partial at wash and elution |
| | (0.58/2.32) | | | | | | |
| C+ | 1:6 | 24 | 600 | 250 | 1000 | 200 | Partial at wash and elution |
| | (0.67/4.2) | | | | | | |
| D+ | 1:8 | 24 | 600 | 250 | 1000 | 200 | Partial at wash and elution |
| | (0.49/3.92) | | | | | | |
| E | 1:6* | 0 | 600 | 600 | 1000 | 200 | No |

*clarified by centrifuging

The results show that at high numbers of cells, the samples clog the purification media but at lower cell counts, very good results are obtained. The number of bacterial cells in each sample per mg of DE analysed is on the higher end of about 2.6×10⁶ cells/mg DE. FIG. 6 further shows the very good yield obtained with this method even though samples A, B, C, and D were not clarified at all. Whole crude cell lysates from bacteria were able to be separated and the desired analyte purified from the mixtures at an extremely high yield (lighter bars in FIG. 6) as compared to samples purified with no DE present (darker bars in FIG. 6).

### Example 6

### Purification of Secreted IgG Antibody from HEK Cells

This example examines how the purification apparatus performs in purifying secreted recombinant proteins from mammalian cells. Much like Examples 3 and 4, the purification apparatus performs very well when DE is included in the purification media when using a Protein A affinity resin. However, in this example instead of adding the recombinant protein to the cells, the recombinant protein is expressed and secreted from the cells. The recombinant protein in this example is secreted IgG antibody.

The purification apparatus was prepared as in prior examples. Particularly, 100 μL of a 50:50 slurry of Protein A resin (Fastback Protein A Resin, Protein Ark, Ltd., Sheffield, UK; Protein A is Staphylococcal Protein A, 41 kDa, recombinant, from Prospec-Tany Technogene Ltd., Rehovot, Ill.) was added to an 96-well microtiter plate (Protein Ark, Ltd., Sheffield, UK). The Protein A resin is a Sepharose® Fast Flow resin (Protein Ark, Ltd., Sheffield, UK) with a ligand density of 3.5 mg Protein A per mL fully hydrated resin (as shipped), with bead size of 60 to 165 μm, and a binding capacity for human IgG antibody of about 30 mg/mL. The resin was resuspended in suspension buffer including distilled water with 0.01% thimerosal water and allowed to normalize at room temperature to create a 50:50 slurry. Then, 100 μL of the 50:50 resin slurry was pipetted into each well. The 96-well plate is commercially available prepackaged with polytetrafluoroethylene (PTFE) membranes at the bottom from Protein Ark, Ltd., Sheffield, UK.

Empty wells of 96-well microtiter plates were loaded with 100 μL of a 50:50 Protein A slurry with or without 100 μL of 50:50 slurry of DE layered on top (Celpure® 300, Imerys Filtration Minerals, Inc., San Jose, Calif., US, about 24 mg DE per well). The 96-well microtiter plates were then allowed to dry at ambient temperature in a positive flow hood.

Transformed human embryonic kidney (HEK 293 6E cells; see Cellosaurus accession number HEK293-EBNA1-6E (RRID:CVCL_HF20); U.S. Pat. No. 8,551,774; see also Jager et al., BMC Proceedings, 9:P40, 2015; Jager et al., BMC Biotechnol., 13:52, 2013; National Research Council (NRC), Canada) cells transiently expressing a secreted form of immunoglobulin G (IgG4, both heavy and light chains) were grown in F17 FreeStyle™ expression medium (ThermoFisher Scientific, Waltham, Mass., US) to confluency of 3×10⁶ cells/mL and collected. Cells were centrifuged and resuspended in F17 media at the indicated cell densities. Various amounts of collected whole cells in media were then added to the wells of the microtiter plates in 600 L, aliquots, including cell densities of 2.5×10⁶ cells/mL, 5×10⁶ cells/mL, 10×10⁶ cells/mL, 15×10⁶ cells/mL, 25×10⁶ cells/mL, and 50×10⁶ cells/mL. The 96-well microtiter plates were then attached to a Grant microplate shaker (Grant Instruments, Cambridgeshire, UK) and shaken at 800 rpm for 30 minutes at ambient temperature. Plates were then transferred to a Beckman Coulter Avanti J-E XP centrifuge equipped with a JS5.3 rotor and centrifuged for 2 minutes at 2000×g at ambient temperature. (Beckman Coulter Life Sciences, Ind., US).

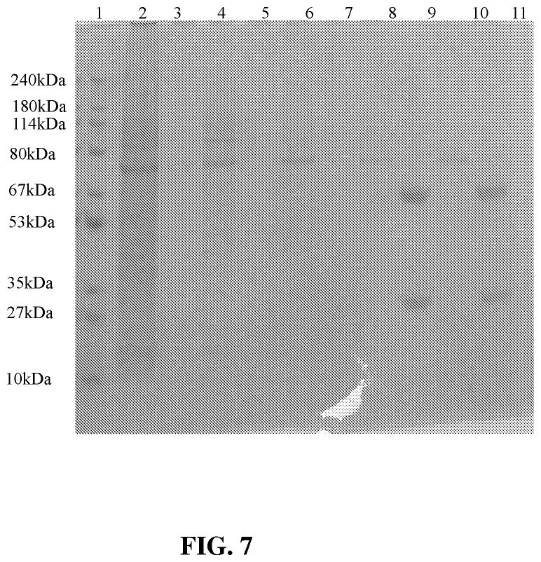

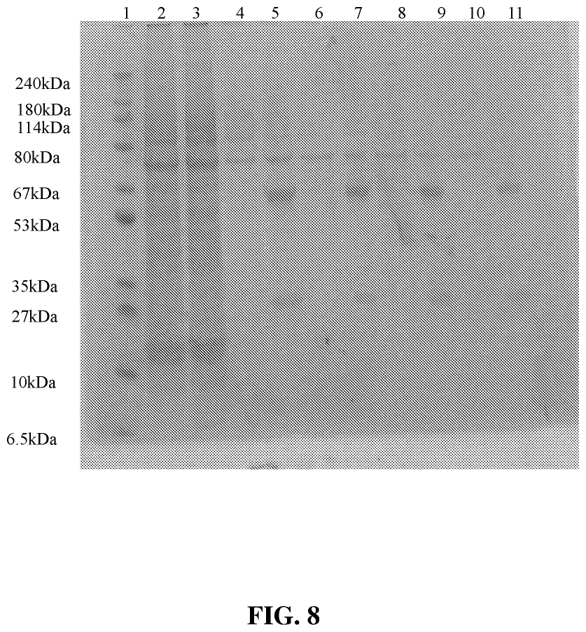

Plates were removed from the centrifuge and samples were washed with 500 μL binding buffer (1.5 M glycine/NaOH, 3 M NaCl, pH 9.0) and centrifuged again at the same speed and for the same time at the same temperature. This wash step was repeated 2 times for each plate. A volume of 200 μL of elution buffer (0.2 M glycine/HCl, pH 2.5) was added to each well after the last wash and the plates again centrifuged under identical conditions. Wells were analysed visually for clogging at each stage (see Table 5) and eluate was examined by reducing SDS-PAGE (see FIGS. 7 and 8). In Table 5, samples marked with a “+” sign indicates the presence of DE in those samples.

For SDS-PAGE analysis 10 μL of each eluate was loaded onto 4-12% SDS-PAGE gels and electrophoresed for 1 hr at room temperature in 2-(N-morpholino)ethanesulfonic acid (MES) buffer. In FIGS. 7 and 8, wells 1 through 11 correspond to the following samples: 1) Elite Pre-Stained Ladders (Protein Ark, Ltd., Sheffield, UK); 2) 25×10⁶ cells/mL wash; 3) 25×10⁶ cells/mL elution; 4) 15×10⁶ cells/mL wash; 5) 15×10⁶ cells/mL wash; 6) 10×10⁶ cells/mL wash; 7) 10×10⁶ cells/ml elution; 8) 5×10⁶ cells/mL wash; 9) 5×10⁶ cells/mL elution; 10) 2.5×10⁶ cells/mL wash; and 11) 2.5×10⁶ cells/mL elution. FIG. 7 results are from wells with no added DE. FIG. 8 results are from wells with approximately 24 mg DE added to each well prior to drying and application of sample, corresponding to the “+” marked samples in Table 5.

TABLE 5

| | | | | | | Post Wash | Post Elution | | |
|---|---|---|---|---|---|---|---|---|---|
| | Cell Density | | | Starting | | Vol. (μl) | Vol. (μl) | | |
| | HEK 293 6E | | | Vol. (μl) | Flowthrough | (500 μl | (200 μl | | |
| | Expressing | | | (mix @ | Volume (μl) | wash buffer) | elution buffer) | | |
| | Secreted IgG | Viability | DE | 800 rpm, | (2000 × g, | (2000 × g, | (2000 × g, | Recovery | |
| Sample | (cells/mL) | (%) | (mg) | 30 min) | 2 min) | 2 min × 2) | 2 min) | (%) | Clogging |
| A | 2.5 × 10⁶ | 70 | 0 | 600 | 600 | 1000 | 200 | 92 | No |
| B | 5 × 10⁶ | 70 | 0 | 600 | 600 | 1000 | 200 | 95 | No |
| C | 10 × 10⁶ | 70 | 0 | 600 | 600 | 1000 | 50 | 0 | Partial at elution |
| D | 15 × 10⁶ | 70 | 0 | 600 | 550 | 1000 | 50 | 0 | Partial at elution |
| E | 25 × 10⁶ | 70 | 0 | 600 | 300 | 60 | 50 | 0 | Partial at wash/elution |
| F | 50 × 10⁶ | 70 | 0 | 600 | 0 | 600 | 50 | 0 | Yes at sample loading |
| A+ | 2.5 × 10⁶ | 70 | 24 | 600 | 600 | 1000 | 200 | 92 | No |
| B+ | 5 × 10⁶ | 70 | 24 | 600 | 600 | 1000 | 200 | 94 | No |
| C+ | 10 × 10⁶ | 70 | 24 | 600 | 600 | 1000 | 200 | 94 | No |
| D+ | 15 × 10⁶ | 70 | 24 | 600 | 600 | 1000 | 200 | 96 | No |
| E+ | 25 × 10⁶ | 70 | 24 | 600 | 600 | 600 | 200 | 0 | Partial at wash |
| F+ | 50 × 10⁶ | 70 | 24 | 600 | 600 | 600 | 200 | 0 | Partial at wash |

The results in Table 5 show that DE rescues wells with no DE from clogging for cell density samples of 25×10⁶ cells/mL and 50×10⁶ cells/mL. This corresponds to a cell per mg DE density of approximately 2.0 to 2.4×10⁶ cells/mg DE. After protein purification through the wells, the maximum cell density that still provided optimal results at this amount of resin and DE for the Protein A plate was observed to be about 15×10⁶ cells/mL, which corresponds to 3.75×10⁴ cells/mg DE (FIGS. 7 and 8). It is also clear from this example that the DE does not interfere substantially with protein recovery or yield.

### Example 7

### Purification of Secreted IgG Antibody from CHO Cells

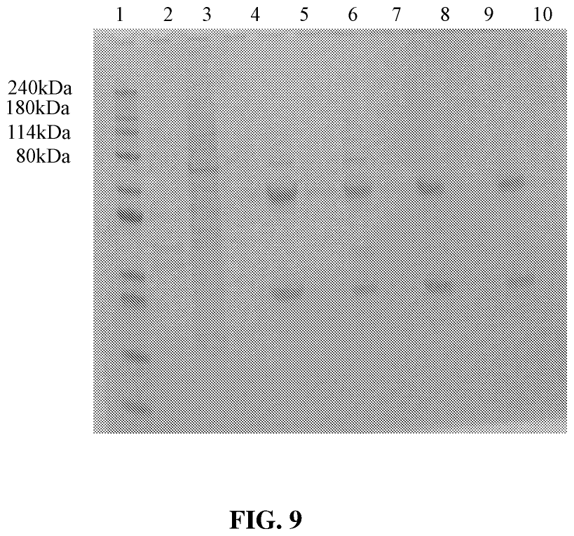

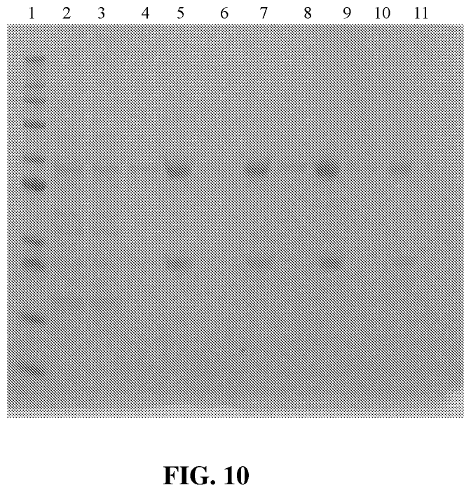

Essentially the same protocol as described in Example 6 was followed in this example, except that instead of HEK cells, Chinese Hamster Ovary (CHO) cells expressing secreted IgG antibody were examined. (CHO cells are from the National Research Council of Canada; see Mellahi et al., Bioproc. Biosys. Eng., 42:711-725, 2019; cumate-inducible GS-CHO cell line expressing rituximab IgG). Purification and sample recovery were as in Example 6. Wells were visually inspected for clogging at each stage and eluates were analyzed by SDS-PAGE as in Example 6. (See, Table 6, below, and FIGS. 9 and 10, respectively). The resin used in this Example was also Protein A as in Example 6. In Table 6, samples marked with a “+” sign indicates the presence of DE in those samples.

In FIGS. 9 and 10, wells 1 through 11 correspond to the following samples: 1) Elite Pre-Stained Ladders; 2) 25×10⁶ cells/mL wash; 3) 25×10⁶ cells/mL elution; 4) 15×10⁶ cells/mL wash; 5) 15x10⁶ cells/mL wash; 6) 10×10⁶ cells/mL wash; 7) 10×10⁶ cells/ml elution; 8) 5×10⁶ cells/mL wash; 9) 5×10⁶ cells/mL elution; 10) 2.5×10⁶ cells/mL wash; and 11) 2.5x10⁶ cells/mL elution. FIG. 9 results are from wells with no added DE. FIG. 10 results are from wells with approximately 24 mg DE added to each well prior to drying and application of sample.

TABLE 6

| | | | | | | Post Wash | Post Elution | | |
|---|---|---|---|---|---|---|---|---|---|
| | Cell Density | | | Starting | | Vol. (μl) | Vol. (μl) | | |
| | CHO Cells | | | Vol. (μl) | Flowthrough | (500 μl | (200 μl | | |
| | Expressing | | | (mix @ | Volume (μl) | wash buffer) | elution buffer) | | |
| | Secreted IgG | Viability | DE | 800 rpm, | (2000 × g, | (2000 × g, | (2000 × g, | Recovery | |
| Sample | (cells/mL) | (%) | (mg) | 30 min) | 2 min) | 2 min × 2) | 2 min) | (%) | Clogging |
| A | 2.5 × 10⁶ | 20 | 0 | 600 | 600 | 1000 | 200 | 95 | No |
| B | 5 × 10⁶ | 20 | 0 | 600 | 600 | 1000 | 200 | 96 | No |
| C | 10 × 10⁶ | 20 | 0 | 600 | 600 | 1000 | 200 | 92 | No |
| D | 15 × 10⁶ | 20 | 0 | 600 | 550 | 1000 | 200 | 93 | No |
| E | 25 × 10⁶ | 20 | 0 | 600 | 300 | 800 | 200 | 0 | Partial at wash |
| F | 50 × 10⁶ | 20 | 0 | 600 | 0 | 800 | 200 | 0 | Yes at sample loading |
| A+ | 2.5 × 10⁶ | 20 | 24 | 600 | 600 | 1000 | 200 | 92 | No |
| B+ | 5 × 10⁶ | 20 | 24 | 600 | 600 | 1000 | 200 | 96 | No |
| C+ | 10 × 10⁶ | 20 | 24 | 600 | 600 | 1000 | 200 | 95 | No |
| D+ | 15 × 10⁶ | 20 | 24 | 600 | 600 | 1000 | 200 | 96 | No |
| E+ | 25 × 10⁶ | 20 | 24 | 600 | 600 | 1000 | 200 | 0 | Partial at wash |
| F+ | 50 × 10⁶ | 20 | 24 | 600 | 600 | 1000 | 200 | 0 | Partial at wash |

As in Example 6, the DE rescues clogged wells for cell density samples of up to 25×10⁶ cells/mL and 50×10⁶ cells/mL, as shown in Table 6. After protein purification, the maximum cell density that provided optimal results for this amount of resin and DE for the Protein A plate is 15×10⁶ cells/mL. (FIGS. 9 and 10). As in the Example 6, it is also clear from this example that the DE does not interfere substantially with protein recovery or yield and in fact markedly improves the recovery as compared to samples purified in purification apparatuses having no DE present.

The breadth and scope of the present disclosure should not be limited by any of the above-described exemplary embodiments, but should be defined only in accordance with the following claims and their equivalents. That is, the above examples are included to demonstrate various exemplary embodiments of the described methods and systems. It will be appreciated by those of skill in the art that the techniques disclosed in the examples represent techniques discovered by the inventor to function well in the practice of the described methods and systems, and thus can be considered to constitute optional or exemplary modes for its practice. However, those of skill in the art will, in light of the present disclosure, appreciate that many changes can be made in these specific embodiments that are disclosed and still obtain a like or similar result without departing from the spirit and scope of the described methods and systems.

What is claimed is:

1. A purification apparatus for purifying a component from a biological sample, said apparatus comprising a housing, wherein each housing comprises the following elements in the following order from top to bottom:
i) a top opening,
ii) a substantially dry purification media comprising diatomaceous earth (DE) and a resin
iii) a membrane, and
iv) a bottom opening, wherein the purification media is in direct contact with the membrane.

2. The purification apparatus of claim 1, wherein the purification media comprises:
iii) a layer comprising a homogenous mixture of said DE and said resin; or
iv) an upper layer and a lower layer, the upper layer comprising said DE and the lower layer comprising said resin, optionally wherein the purification media comprises a boundary area between the upper layer and the lower layer, said boundary area comprising a mixture of DE and resin.

3. The purification apparatus of claim 1, wherein the component is a protein, and optionally wherein the purification media comprises one or more wetting agents.

4. The purification apparatus of claim 1, wherein:
i) the resin is not an affinity resin; or
ii) the resin is an affinity resin and comprises one or more of Protein A, Protein G, Protein L, an antibody or antigen binding fragment thereof, heparin, or lectin; or
iii) the resin is an immobilized metal affinity chromatography (IMAC) resin, preferably comprising one or more of: Zn²⁺, Cu²⁺, Cd²⁺, Hg²⁺, Co²⁺, Ni²⁺, and Fe²⁺.

5. The purification apparatus of claim 1, wherein the biological sample comprises eukaryotic cells, and wherein DE is present in said purification media in an amount of from about 1 mg DE per 6.25×10⁴ eukaryotic cells to about 1 mg DE per 1.25×10⁶ eukaryotic cells, preferably from about 1 mg DE per 24×10⁴ eukaryotic cells to about 1 mg DE per 37.5×10⁴ eukaryotic cells, preferably wherein said cells are mammalian cells.

6. The purification apparatus of claim 1, wherein the DE comprises:
a) about 84.1 mg/kg aluminum, about 52.5 mg/kg calcium, about 50.5 mg/kg magnesium, about 20.0 mg/kg iron, about 10.5 mg/kg zinc, about 0.8 mg/kg copper, about 0.6 mg/kg antimony, about 0.7 mg/kg manganese, and about 0.2 mg/kg chromium; or
b) about 6.2 mg/kg magnesium, about 2.8 mg/kg iron, about 0.6 mg/kg copper, and about 0.2 mg/kg manganese; or
c)

| | % |
|---|---|
| SiO₂ | 98.65 |
| Al₂O₃ | 0.60 |
| Fe₂O₃ | 0.27 |
| Na₂O | 0.14 |
| K₂O | 0.10 |
| MgO | 0.08 |
| CaO | 0.08 |
| TiO₂ | 0.03 |
| P₂O₅ | 0.03 |

7. The purification apparatus of claim 1, wherein the DE is produced by a process that
comprises:
washing the DE with water,
heating the washed DE to about 1000° C. to calcinate the DE, washing the calcinated DE in acid to create acid-washed DE, and heating the acid-washed DE in water to 200° C. to create dried DE.

8. The purification apparatus of claim 1, wherein the membrane comprises, preferably consists of, polytetrafluoroethylene (PTFE).

9. The purification apparatus of claim 1, wherein the housing tapers to a tip at the bottom opening below the membrane.

10. The purification apparatus of claim 1, wherein the housing is a round tube that is optionally comprised of a plastic polymer, preferably wherein the housing holds a liquid volume of between about 0.6 mL and about 2.0 mL.

11. The purification apparatus of claim 1, wherein the apparatus is:
i) a microtiter plate comprising 8, 24, 96, 384, or 1536 housings per plate, and wherein the plate and the housings are comprised of a plastic polymer;
ii) a spin column;
iii) a 24-well plate; or
iv) a 96-well microliter plate.

12. A method of preparing a purification apparatus for purifying a component from a biological sample, said method comprising:
D) providing a housing comprising a top opening, a bottom opening, and a membrane positioned between said top opening and said bottom opening;
E) adding to the housing a purification media comprising diatomaceous earth (DE) and a resin, wherein the purification media is positioned between the membrane and the top opening; and
F) drying the purification media.

13. The method of claim 12, wherein the DE and resin are added to the housing through the top opening in the form of wet slurries, preferably wherein the resin is added in the form of a 50:50 slurry comprising 50% fully hydrated resin and an aqueous solution, and wherein the DE is added in the form of a 50:50 slurry comprising 50% DE and an aqueous solution.

14. A method of purifying a component from a biological sample, said method comprising:
C) applying a biological sample to a top opening of a purification apparatus for purifying a component from a biological sample, wherein the purification apparatus comprises:
a housing comprising a top opening, a bottom opening, and a membrane positioned between said top opening and said bottom opening; and
a purification media comprising diatomaceous earth (DE) and a resin, wherein the purification media is positioned between the membrane and the top opening, and wherein the purification media is substantially dry;
D) mixing the sample in liquid buffer with the purification media in the housing.

15. The method of claim 14, which further comprises:
C) clearing liquid from the apparatus via the bottom opening; and/or
D) washing said purification media one or more times; and/or
E) eluting the purified component from the apparatus, and optionally collecting the purified biological sample as it exits the bottom opening.

16. The method of claim 15, wherein one or more of steps C) to E) further comprises subjecting the housing comprising the sample and purification media liquid mixture to centrifugation, applying a positive pressure to the top opening of the apparatus, or applying vacuum pressure to the bottom opening of the purification apparatus.

17. The method of claim 14, wherein:
i) the resin is an affinity resin, and wherein the affinity resin comprises Protein A, Protein G, Protein L; or
ii) the resin is an immobilized metal affinity chromatography (IMAC) resin.

18. The method of claim 14, wherein the biological sample comprises whole cells, a cell extract or a cell lysate, preferably wherein the cells, cell extract, or cell lysate comprise(s) recombinantly expressed proteins, preferably wherein the cells are eukaryotic cells or prokaryotic cells.

19. The method of claim 18, wherein:
i) the cells are eukaryotic cells, preferably mammalian cells, wherein the amount of DE present in the purification media is from approximately 1 mg DE per 6.25×10⁴ eukaryotic cells to about 1 mg DE per 1.25×10⁶ eukaryotic cells, preferably from about 1 mg DE per 24×10⁴ eukaryotic cells to about 1 mg DE per 37.5×10⁴ eukaryotic cells; or
ii) the cells are prokaryotic cells, preferably bacterial cells, wherein the cells are lysed prior to applying the sample to the top opening of the housing, and wherein DE is present in the purification media in an amount of less than approximately 1 mg DE per 2.5×10⁶ lysed cells.
